SEQUENCE LISTING (40 sequences)

SEQ ID NO: 1 - protein, 9 aa, Artificial Sequence
  MARK3 splice variant antigen
RNMSFRFIK

SEQ ID NO: 2 - protein, 9 aa, Artificial Sequence
  NBPF9 splice variant antigen
SSFYALEEK

SEQ ID NO: 3 - protein, 10 aa, Artificial Sequence
  PARD3 splice variant antigen
SQLDFVKTRK

SEQ ID NO: 4 - protein, 9 aa, Artificial Sequence
  ZC3HAV1 splice variant antigen
LTMAVKAEK

SEQ ID NO: 5 - protein, 9 aa, Artificial Sequence
  YAF2 splice variant antigen
VIVSASRTK

SEQ ID NO: 6 - protein, 9 aa, Artificial Sequence
  CAMKK1 splice variant antigen
VTSPSRRSK

SEQ ID NO: 7 - protein, 9 aa, Artificial Sequence
  LRR1 splice variant antigen
SLPRFGYRK

SEQ ID NO: 8 - protein, 10 aa, Artificial Sequence
  ZNF670 splice variant antigen
SCVSPSSELK

SEQ ID NO: 9 - protein, 9 aa, Artificial Sequence
  ZC3HAV1 splice variant antigen
LTMAVKAEK

SEQ ID NO: 10 - protein, 9 aa, Artificial Sequence
  GRINA splice variant antigen
SIRQAFIRK

SEQ ID NO: 11 - protein, 9 aa, Artificial Sequence
  MZF1 splice variant antigen
KWPPATETL

SEQ ID NO: 12 - protein, 10 aa, Artificial Sequence
  MZF1 splice variant antigen
AVFDRKSDAK

SEQ ID NO: 13 - protein, 9 aa, Artificial Sequence
  EBV F12 antigen
IVTDFSVIK

SEQ ID NO: 14 - protein, 329 aa, Artificial Sequence
  CD86
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 15 - protein, 193 aa, Artificial Sequence
  CD70
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 16 - protein, 254 aa, Artificial Sequence
  CD137L
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 17 - protein, 365 aa, Artificial Sequence
  HLA-A11 with HLA-A11 leader sequence
MAVMAPRTLLLLLSGALALTQTWAGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 18 - protein, 504 aa, Artificial Sequence
  MARK3 SC HLA-A11
MSRSVALAVLALLSLSGLEARNMSFRFIKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 19 - protein, 20 aa, Artificial Sequence
  leader sequence
MSRSVALAVLALLSLSGLEA

SEQ ID NO: 20 - protein, 15 aa, Artificial Sequence
  GS linker 1
GGGGSGGGGSGGGGS

SEQ ID NO: 21 - protein, 99 aa, Artificial Sequence [sequence omitted]

SEQ ID NO: 22 - protein, 20 aa, Artificial Sequence
  GS linker 2
GGGGSGGGGSGGGGSGGGGS

SEQ ID NO: 23 - protein, 341 aa, Artificial Sequence
  HLA-A11
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 24 - protein, 504 aa, Artificial Sequence
  NBPF9 SC HLA-A11
MSRSVALAVLALLSLSGLEASSFYALEEKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 25 - protein, 505 aa, Artificial Sequence
  PARD3 SC HLA-A11
MSRSVALAVLALLSLSGLEASQLDFVKTRKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 26 - protein, 504 aa, Artificial Sequence
  ZC3HAV1 SC HLA-A11
MSRSVALAVLALLSLSGLEALTMAVKAEKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 27 - protein, 504 aa, Artificial Sequence
  YAF2 SC HLA-A11
MSRSVALAVLALLSLSGLEAVIVSASRTKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 28 - protein, 504 aa, Artificial Sequence
  CAMKK1 SC HLA-A11
MSRSVALAVLALLSLSGLEAVTSPSRRSKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 29 - protein, 504 aa, Artificial Sequence
  LRR1 SC HLA-A11
MSRSVALAVLALLSLSGLEASLPRFGYRKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 30 - protein, 505 aa, Artificial Sequence
  ZNF670 SC HLA-A11
MSRSVALAVLALLSLSGLEASCVSPSSELKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 31 - protein, 504 aa, Artificial Sequence
  GRINA SC HLA-A11
MSRSVALAVLALLSLSGLEASIRQAFIRKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 32 - protein, 504 aa, Artificial Sequence
  MZF1 SC HLA-A24
MSRSVALAVLALLSLSGLEAKWPPATETLGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWEPSSQPTVPIVGIIAGLVLLGAVITGAVVAAVMWRRNSSDRKGGSYSQAASSDSAQGSDVSLTACKV

SEQ ID NO: 33 - protein, 505 aa, Artificial Sequence
  EBV F12 SC HLA-A11
MSRSVALAVLALLSLSGLEAAVFDRKSDAKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 34 - protein, 504 aa, Artificial Sequence
  EBV F28 SC HLA-A11
MSRSVALAVLALLSLSGLEAIVTDFSVIKGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGRCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWELSSQPTIPIVGIIAGLVLLGAVITGAVVAAVMWRRKSSDRKGGSYTQAASSDSAQGSDVSLTACKV

SEQ ID NO: 35 - protein, 341 aa, Artificial Sequence
  HLA-A02
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 36 - protein, 504 aa, Artificial Sequence
  CMV pp65 SC HLA-A02
MSRSVALAVLALLSLSGLEANLVPMVATVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 37 - protein, 504 aa, Artificial Sequence
  cancer SC HLA-A02
MSRSVALAVLALLSLSGLEAFLLDGSANVGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGGGGSGSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMAAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWEPSSQPTIPIVGIIAGLVLFGAVITGAVVAAVMWRRKSSDRKGGSYSQAASSDSAQGSDVSLTACKV

SEQ ID NO: 38 - protein, 9 aa, Artificial Sequence
  Antigen
NLVPMVATV

SEQ ID NO: 39 - protein, 9 aa, Artificial Sequence
  antigen
FLLDGSANV

SEQ ID NO: 40 - protein, 365 aa, Artificial Sequence [sequence omitted]

(19) United States
(12) Patent Application Publication
(10) Pub. No.: US 2024/0060042 A1
(43) Pub. Date: Feb. 22, 2024

(54) ARTIFICIAL ANTIGEN-PRESENTING CELL

(71) Applicant: National University of Singapore, Singapore (SG)

(72) Inventor: Teck Ho Raymond LEE, Singapore (SG)

(21) Appl. No.: 18/259,020

(22) PCT Filed: Dec. 22, 2021
(86) PCT No.: PCT/SG2021/050818

§ 371 Date: Jun. 22, 2023

(30) Foreign Application Priority Data
Dec. 22, 2020 (SG) 10202012952P
Jul. 21, 2021 (SG) 10202107937Q

(51) Int. Cl. C12N 5/0783; G01N 33/50; C12N 15/86; A61K 35/17

(52) U.S. Cl. CPC C12N 5/0636; G01N 33/505; C12N 15/86; A61K 35/17; C12N 2502/99; C12N 2501/2307; C12N 2501/2315; C12N 2501/2321; C12N 2740/15043; C12N 2502/30

(57) ABSTRACT

The invention relates to an artificial antigen-presenting cell (aAPC) comprising at least one immune stimulatory ligand and co-stimulatory ligands comprising or consisting of CD86, CD70 and CD137L, methods of preparing an aAPC and methods of inducing proliferation of an immune cell or expanding a population of immune cells. The invention also relates to methods for inducing an immune response or treating a medical condition in a subject. The invention further relates to methods of identifying an antigenic peptide or method of identifying or detecting the presence of an immune cell that recognizes an antigen.

### FIELD OF INVENTION

The invention relates generally to immunology. In particular, the invention relates to an artificial antigen-presenting cell (aAPC) and methods of inducing proliferation of an immune cell or expanding a population of immune cells.

### BACKGROUND

Adoptive T cell therapy is a promising cell therapy that involves removal of T cells from a subject, ex vivo processing and subsequent infusion of the T cells back into a subject. The technique may involve genetic modification of the T cells to enhance their specificity for a particular antigen, such as by expression of a chimeric antigen receptor (CAR). It may also involve selection of T cells that are specific for a particular antigen.

Currently it is challenging to generate antigen-specific T cells in vitro as well as to obtain sufficient quantities of antigen-specific T cells. Typically, the generation or expansion of antigen-specific T cells would require the use of dendritic cells or feeder cells that are derived from the same donor. This limits the ability to expand or obtain large amount of antigen-specific T cells due to limited availability of these dendritic cells and feeder cells.

Artificial antigen-presenting cells (aAPCs) have been constructed using either cell based systems or synthetic scaffolds. For expansion of antigen-specific T cells, these aAPCs present peptide/HLA complexes that can be recognized by antigen-specific T cells, and immunostimulatory molecules (such as co-stimulatory ligands) that enhance proliferation or augment phenotype of antigen-specific T cells.

Limitation of current T cell stimulation/expansion methods using current aAPC revolves around the uncertainty of how combinations of co-stimulatory ligands are required to induce and stimulate the expansion of antigen-specific T cells. This uncertainty is further compounded by the state that T cells are in during antigen stimulation. In some cases, over-stimulation of T cells leads to an exhausted immunophenotype and it is more difficult to induce the proliferation of exhausted T cells.

Accordingly, it is generally desirable to overcome or ameliorate one or more of the above-mentioned difficulties.

### SUMMARY

Disclosed herein is an artificial antigen-presenting cell (aAPC) comprising at least one immune stimulatory ligand and co-stimulatory ligands comprising or consisting of CD86, CD70 and CD137L, wherein the aAPC is capable of inducing proliferation and/or expanding an immune cell population when contacted with the aAPC.

Disclosed herein is an isolated nucleic acid encoding at least one immune stimulatory ligand and co-stimulatory ligands comprising or consisting of CD86, CD70 and CD137L.

Disclosed herein is a vector comprising an isolated nucleic acid as defined herein.

Disclosed herein is a method of preparing an aAPC as defined herein, wherein the method comprises transfecting or transducing a mammalian cell with an isolated nucleic acid or one or more vectors as defined herein.

Disclosed herein is a method for inducing proliferation of an immune cell, said method comprising contacting the immune cell with an aAPC as defined herein, thereby inducing proliferation of the immune cell.

Disclosed herein is a method for expanding a population of antigen-specific immune cells, said method comprising contacting the population of immune cells with an aAPC as defined herein, thereby expanding the population of the antigen-specific immune cells.

Disclosed herein is a method of generating one or more populations of antigen-specific immune cells, said method comprising contacting a population of immune cells with one or more aAPC as defined herein to generate one or more populations of antigen-specific immune cells.

Disclosed herein is an aAPC as defined herein for use as a medicament.

Disclosed herein is a method for inducing an immune cell response to an antigen in a subject, the method comprising administering the aAPC as defined herein to the subject, wherein the aAPC induces proliferation of an immune cell specific for the antigen, thereby inducing an immune cell response to the antigen in the subject.

Disclosed herein is a method of treating a medical condition in a subject, the method comprising: a) expanding a population of immune cells that has been isolated from a subject by contacting the population of immune cells with the aAPC as defined herein; and b) administering the expanded population of immune cells to the subject to treat the medical condition in the subject.

Disclosed herein is a method of identifying an antigenic peptide in a subject, the method comprising: a) contacting an aAPC as defined herein with a population of immune cells that has been obtained from the subject, wherein the aAPC is loaded with a candidate peptide or comprises a recombinant nucleic acid encoding a candidate peptide; and b) detecting a population of immune cells that recognizes the candidate peptide, thus identifying the candidate peptide as an antigenic peptide.

Disclosed herein is a method for identifying or detecting the presence of an immune cell that recognizes an antigen, said method comprising contacting said immune cell with one or more aAPCs as defined herein that presents said antigen, and identifying or detecting the presence of a population of immune cells that recognizes said antigen.

### BRIEF DESCRIPTION OF DRAWINGS

Embodiments of the present invention are hereafter described, by way of non-limiting example only, with reference to the accompanying drawings in which:

FIG. 1: Cell surface expression of co-stimulatory ligands and accessory molecules on K562 (Butler MO, et al. 2007).

FIG. 2 is a diagram of an artificial antigen-presenting cell (aAPC) in contact with a T cell. Artificial antigen-presenting cell comprising at least one immune stimulatory ligand and co-stimulatory ligand. Co-stimulation of T-cell which leads to T-cell activation occurs through binding of one or more co-stimulatory ligands present on the artificial antigen-presenting cell.

FIG. 3 shows a lentiviral construct consisting of HLA molecule (HLA-A11), CD86, CD70 and CD137L (4-1BBL).

FIG. 4 is a graphical representation of FACS data showing generation of CD8+ antigen-specific T cells for MARK3 splice variant peptide (SVP). MARK3-specific T cells were generated by co-culturing CD8+ T cells with monocyte-derived dendritic cells (moDCs) from healthy donor PBMC (donor HSA38). After 11 days of co-culture, MARK3-specific T cells were detected using MARK3 tetramers labeled with both PE and APC. Stimulation of CD8+ T cells with monocyte-derived dendritic cells (moDCs) loaded with MARK3 SVP leads to the appearance of MARK3 SVP-specific T cells (0.12% of CD8+ T cells; middle graph, moDC Stim)); re-stimulation with artificial antigen-presenting cells (aAPCs) loaded with MARK3 SVP leads to the further enrichment of MARK3 SVP specific T cells (0.71% of CD8+ T cells; right graph, aAPC ReStim).

FIG. 5 is a graphical representation of FACS data showing generation of CD8+ antigen-specific T cells for LRR1 and GRINA SVPs, top and bottom panels, respectively). Antigen-specific T cells were generated by co-culturing CD8+ T cells with moDCs from healthy donor PBMC (donors HSA27 and HSA38 for LRR1 and GRINA, respectively). In this case, after 10 days of co-culture, antigen-specific T cells for LRR1 and GRINA SVPs were detected using tetramers that were loaded with LRR1 and GRINA peptides and labeled with both PE and APC. Antigen-specific T cells generated after this initial stimulation were 0.008% and 0.025 of total CD8+ T cells for LRR1 and GRINA, respectively (middle column, moDC Stim). These cells were then restimulated with artificial APCs loaded with peptide for a further 11 days. Re-stimulation with artificial APCs led to the further expansion of antigen-specific T cells (0.042% and 0.26% of CD8+ T cells for LRR1 and GRINA, respectively; right column, aAPC ReStim).

FIG. 6 is a graphical representation of FACS data for antigen-specific CD8+ T cells generated for MARK3 SVPs. MARK3 tetramers labeled with both PE and APC were used to detect MARK3-specific T cells. Unstimulated PBMCs from healthy donor (HSA12) do not contain antigen-specific CD8+ T cells that recognize MARK3 SVP. Middle panel (aAPC Stim-1) shows the appearance of MARK3 SVP-specific T cells after stimulation of naïve CD8+ T cells with aAPCs that have been loaded with MARK3 SVP (0.11% of CD8+ T cells). Right panel (aAPC Stim-2) shows further expansion of MARK3 SVP-specific T cells after a second round of stimulation with aAPCs loaded with MARK3 SVP (0.56% of CD8+ T cells).

FIG. 7: Artificial APC construct expressing MARK3 SVP single chain (SC) HLA and co-stimulatory ligands.

FIG. 8 is a graphical representation of FACS data showing generation of antigen-specific T-cells for LRR1 and MARK3 from naïve CD8+ T cells isolated and co-cultured with SC HLA-A11 aAPC. Tetramers loaded with LRR1 and MARK3 peptide, labelled with PE and APC, respectively, were used to detect antigen-specific T cells for LRR1 and MARK3 SVP. Naive CD8+ T cells were isolated from a healthy donor (HSA38) and co-cultured with LRR1 SC HLA-A11 aAPC, MARK3 SC HLA-A11 aAPC, or both LRR1 and MARK3 SC HLA-A11 aAPCs. Antigen-specific T cells against LRR1 and MARK3 were generated when naïve CD8+ T cells were co-cultured with the respective SC HLA-A11 aAPC. For example, LRR1-specific T cells were generated when naïve CD8+ T cells were co-cultured with LRR1 SC HLA-A11 aAPC (0.13% LRR1 Tetramer: PE positive cells). Furthermore, when both LRR1 and MARK3 SC HLA-A11 aAPCs were co-cultured with naïve CD8+ T cells, antigen-specific T cells for LRR1 and MARK3 could be identified (0.55% LRR1-specific T cells and 0.071% MARK3-specific T cells).

FIG. 9 is a graphical representation of FACS data showing SC aAPC expansion of EBV-Specific T cells. A) Two different HLA-A11 epitopes (F12 and F28) are present in EBV EBNA 3B. Tetramers loaded with EBV F12 and EBV F28 peptide, labelled with PE and APC, respectively, were used to detect antigen-specific T cells. Ex-vivo tetramer staining of PBMC from donor HSA29 shows that he/she had been previously exposed to this virus and has T cells that recognise both of these epitopes (0.072% EBV F12 Tetramer: PE positive CD8+ cells and 1.21% EBV F28 Tetramer: APC positive CD8+ cells). B) Total CD8+ T cells were isolated from HSA29 and co-cultured with EBV F12 SC HLA-A11 aAPC, EBV F28 SC HLA-A11 aAPC, and both EBV F12 SC HLA-A11 aAPC and EBV F28 SC HLA-A11 aAPC. Antigen-specific T cells to both of these antigens were further expanded when co-cultured with the SC HLA-A11 aAPC. For example, EBV F28-specific T cells were generated when total CD8+ T cells were co-cultured with EBV F28 SC HLA-A11 aAPC (6.6% EBV F28 Tetramer: APC positive cells). Furthermore, when both EBV F12 and EBV F28 SC HLA-A11 aAPCs were co-cultured with CD8+ T cells, antigen-specific T cells for EBV F12 and EBV F28 were further expanded (0.14% EBV F12-specific T cells and 4.18% EBV F28-specific T cells).

FIG. 10 is a graphical representation of FACS data showing detection of T cell responses to cancer antigen MARK3 in gastric cancer patients GC43 and SC020. To determine whether gastric cancer patients have T cell responses to MARK3 SVP, PBMCs (GC43) or CD8+ T cells (SC020) were co-cultured with the MARK3 SC aAPC or LRR SC aAPC (irrelevant target). MARK3-specific T cells were identified using tetramers labelled with PE and loaded with MARK3 peptide. CD8+ T cells are identified by staining with CD8 antibody labeled with APC. MARK3 tetramer staining and CD8 expression are shown, and quadrants in the FACs plots show the number of cells expressing different levels of CD8 and labeling by the MARK3 tetramer. There is a greater frequency of MARK3-specific T cells (upper right quadrant) observed for the samples co-cultured with the MARK3 SC aAPC (0.11% and 0.22% for GC43 and SC020, respectively) compared to LRR1 SC aAPC (0% and 0.10% for GC43 and SC020, respectively). An immune response to the MARK3 cancer antigen can be detected in these two patients.

FIG. 11 is a graphical representation of FACS data showing generation of MARK3-specific T cells from HLA-mismatched donor. Total CD8+ T cells were isolated from a HLA-A11 negative healthy donor and co-cultured with MARK3 SC HLA-A11 aAPC. Antigen-specific T cells were identified by staining with tetramers that had been loaded with MARK3 peptide or an irrelevant peptide (GRINA). T cells that were specific for MARK3 peptide were identified by staining the sample with HLA-A11 tetramers that were loaded with MARK3 peptide (tetramers were labelled with PE or APC) or PE-labeled tetramer loaded with MARK3 peptide and APC-labeled tetramer loaded with GRINA peptide, shown in the left and right FACS data, respectively. Group of CD8+ T cells that specifically recognizes MARK3 peptide is shown by dotted box; these cells are stained only by tetramers loaded with MARK3 peptide (FACS data on right), whereas CD8+ T cells that recognize HLA-A11 are shown by the dashed box. These CD8+ T cells are stained by HLA-A11 tetramers irrespective of the peptide that is bound by the HLA-A11 tetramer (both FACs data).

FIG. 12 shows selective expansion of antigen-specific T cells using SC HLA aAPCs with HLA mutations that affect CD8 binding. PE- and APC-labelled dextramers loaded with MARK3 and EBV F28 peptides, respectively, were used to identify the antigen specificity of CD8+ T cells after T cell/aAPC co-culture for 7 days. A) is a graphical representation of FACS data showing dextramer staining for CD8+ T cells for co-cultures of naïve CD8+ T cells (isolated from HSA60 and HSA66) with MARK3 SC aAPC and its HLA binding variants (227 m and 245 m) showing presence of MARK3-specific T cells. MARK3-specific T cells that are stained only with MARK3 peptide loaded dextramer are shown by the box. B) is a graphical representation of FACS data showing the distribution of the staining intensity for the MARK3 dextramer of MARK3-specific T cells in HSA60 and HSA66. Geometric mean of the MARK3 dextramer staining intensity for the MARK3-specific T cells is indicated in the figure. The majority of MARK3-specific T cells generated in HSA66 (HLA mismatched) show greater MARK3 dextramer staining intensity compared to MARK3-specific T cells generated in HSA60 (HLA matched), suggesting that T cells generated in HLA mismatched donor may have higher affinity/avidity for its target. Additionally, MARK3-specific CD8+ T cells generated using the MARK3 227 m SC HLA aAPC show the greatest MARK3 dextramer staining intensity.

FIG. 13 is a series of graphical representations showing the phenotype of CD8+ T cells after co-culture with SC HLA aAPCs and its variants. Phenotype of CD8+ T cells was assessed after 7 days of co-culture using a BV711-labelled antibody recognizing CD25, a marker expressed by activated T cells. This was used to determine the phenotype of CD8+ T cells after T cell/aAPC co-culture. The distribution of T cells expressing different levels of the activation marker CD25 in different co-cultures is shown. Horizontal bars show the percentage of CD8+ T cells that express low (CD25−) or high (CD25+) levels of CD25. Naïve CD8+ T cells from the same donor co-cultured with the MARK3 227 m SC aAPC show a greater percentage of CD8+ T cells that have low expression of CD25 (CD25−) cells compared to MARK3 SC aAPC. For example, CD25− CD8+ T cells are present at 46% and 19.3% in naïve CD8+ T cells from HSA52 co-cultured with MARK3 227 m SC aAPC and MARK3 SC aAPC, respectively.

### DETAILED DESCRIPTION

The present specification teaches an artificial antigen-presenting cell (aAPC) comprising at least one immune stimulatory ligand, wherein the aAPC is capable of inducing proliferation and/or expanding an immune cell population when contacted with the aAPC. The aAPC may comprise co-stimulatory ligands comprising or consisting of CD86, CD70 and CD137L. The aAPC may comprise one or more antigens that are presented on the surface by the at least one immune stimulatory ligand.

The present invention is predicated on the finding that an aAPC expressing at least one immune stimulatory ligand and at least the co-stimulatory ligands, CD86, CD70 and CD137L, is highly effective in inducing and/or stimulating the expansion of an immune cell population as compared to monocyte derived dendritic cells. The induction and/or stimulation of these cells may be further optimized by expressing the at least one immune stimulatory ligand as a fusion protein with an antigen to constitutively present the antigen on the surface of the aAPC. The immune stimulatory ligand may also be modified to have attenuated binding affinity to CD8, which may generate less-differentiated T cells that may persist better in vivo.

In one embodiment, there is provided an artificial antigen-presenting cell (aAPC) comprising at least one immune stimulatory ligand and co-stimulatory ligands comprising or consisting of CD86, CD70 and CD137L, wherein the aAPC is capable of inducing proliferation and/or expanding an immune cell population when contacted with the aAPC.

In one embodiment, the aAPC is capable of inducing proliferation and/or expanding a specific immune cell population when contacted with the aAPC. The specific immune cell population may be specifically recognised by the at least one immune stimulatory ligand that is present on the aAPC.

The phrase “at least one immune stimulatory ligand” may, for example, refer to 1, 2, 3, 4, 5, 6, 7, 8, 9, 10 or more immune stimulatory ligands.

As used herein, the term “artificial antigen-presenting cell (aAPC)” refers to an artificially produced antigen-presenting cell and can be a non-immune cell (such as a tumour or fibroblast cell) modified to express immune molecules such as immune stimulatory ligands (e.g. MHC class I or II molecules) with other accessory molecules (co-stimulatory ligands and/or adhesion molecules). The aAPC can also be modified to express one or more antigens. The one or more antigens may be expressed as fusion proteins with one or more immune stimulatory ligands. An aAPC of the present invention can also be a vesicle, e.g.: a liposome, having a lipid bilayer membrane resembling the lipid bilayer of a naturally occurring cell and can include a nucleic acid encoding immune molecules such as immune stimulatory ligands (e.g.: MHC class I or II molecules) with other accessory molecules (co-stimulatory ligands and/or adhesion molecules). The aAPC of the present invention can also include synthetic scaffolds (such as dextran scaffolds) that can comprise immune molecules such as immune stimulatory ligands (e.g.: MHC class I or II molecules) with other accessory molecules (co-stimulatory ligands and/or adhesion molecules).

By the term “stimulation” is meant a primary response induced by binding of a stimulatory molecule (e.g.: a TCR/CD3 complex) with its cognate ligand (i.e.: stimulatory ligand) thereby mediating a signal transduction event, such as, but not limited to, signal transduction via the TCR/CD3 complex. Stimulation can mediate altered expression of certain molecules, such as down-regulation of TGF-β, and/or reorganization of cytoskeletal structures, and the like.

A “stimulatory ligand” or “immune stimulatory ligand” as used herein, means a ligand that when present on an antigen-presenting cell (e.g.: an aAPC, a dendritic cell, a B-cell, and the like) can specifically bind with a cognate binding partner (referred to herein as a “stimulatory molecule”) on a T cell, thereby mediating a primary response by the T cell, including, but not limited to, activation, initiation of an immune response, proliferation, and the like. Stimulatory ligands are well-known in the art and encompass, for example, an MHC (Class I or Class II) molecule loaded with an antigen (such as a peptide).

A “stimulatory molecule” as the term is used herein, means a molecule on a T cell that specifically binds with a cognate stimulatory ligand present on an antigen-presenting cell (e.g.: an aAPC of the invention, among others).

“Loaded” with an antigen, as used herein, refers to, for example, presentation of an antigen by an MHC molecule or immune stimulatory ligand on the surface of an aAPC. The antigen may be an endogenous or exogenous antigen. An endogenous antigen may, for example, be encoded by a recombinant nucleic acid in the aAPC. An endogenous antigen may alternatively be encoded as a fusion protein with, for example, an MHC molecule. The antigen can also be an exogenous antigen that is loaded by pulsing the aAPC with the exogenous antigen.

“Co-stimulatory ligand,” as the term is used herein, includes a molecule on an antigen-presenting cell (e.g.: an aAPC, dendritic cell, B cell, and the like) that specifically binds a cognate co-stimulatory molecule on a T cell, thereby providing a signal which, in addition to the primary signal provided by, for instance, binding of a TCR/CD3 complex with an MHC molecule loaded with peptide, mediates a T cell response, including, but not limited to, proliferation, activation, differentiation, and the like. A co-stimulatory ligand can include, but is not limited to, CD7, B7-1 (CD80), B7-2 (CD86), PD-L1, PD-L2, CD137L (4-1BBL), OX40L, inducible Co-Stimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as, but not limited to, CD27, CD28, 4-1BB, OX40, CD30, CD40, PD-1, ICOS, lymphocyte function-associated antigen-1 (LFA-1), CD2, D7, LIGHT, NKG2C, B7-H3, and a ligand that specifically binds with CD83.

A “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules include, but are not limited to a CD3, CD28 and CD137 (4-1BB) molecule.

“Activation”, as used herein, refers to the state of a T cell that has been sufficiently stimulated to induce detectable cellular proliferation. Activation can also be associated with induced cytokine production, and detectable effector functions. The term “activated T cells” refers to, among other things, T cells that are undergoing cell division.

By the term “specifically binds,” as used herein, is meant an antibody, or a ligand, which recognizes and binds with a cognate binding partner protein (e.g.: a stimulatory and/or co-stimulatory molecule present on a T cell) present in a sample, but which antibody or ligand does not substantially recognize or bind other molecules in the sample.

The term “antibody,” as used herein, may refer to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. It can also refer to an “antibody fragment”.

The term “antibody fragment” may refer to at least one portion of an antibody that retains the ability to specifically interact with an epitope of an antigen. Examples of antibody fragments include, but are not limited to, Fab, Fab′, F(ab′)2, Fv fragments, scFv antibody fragments, disulfide-linked Fvs (sdFv), a Fd fragment consisting of the VH and CHI domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody.

The terms “peptide” “polypeptide” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, or a combination thereof.

In one embodiment, the aAPC is capable of activating an immune cell population when contacted with the aAPC.

In one embodiment, the aAPC comprises at least one immune stimulatory ligand and co-stimulatory ligands comprising or consisting of CD86, CD70 and CD137L.

In one embodiment, the aAPC comprises at least one immune stimulatory ligand and co-stimulatory ligands comprising or consisting of CD86, CD70, CD137L and CD80.

The aAPC may further comprise one or more adhesion molecules such as ICAM1/CD54 and/or LFA3/CD58.

In one embodiment, the immune stimulatory ligand is a human immune stimulatory ligand. The co-stimulatory ligands may be human co-stimulatory ligands. These may comprise or consist of human CD86, human CD70 and human CD137L. Alternatively, these may comprise or consist of human CD86, human CD70, human CD137L and human CD80.

In one embodiment, there is provided an aAPC engineered to express at least one immune stimulatory ligand and co-stimulatory ligands comprising or consisting of CD86, CD70 and CD137L, wherein the aAPC is capable of inducing proliferation and/or expanding an immune cell population when contacted with the aAPC.

In one embodiment, there is provided an aAPC engineered to express at least one immune stimulatory ligand and co-stimulatory ligands comprising or consisting of CD86, CD70, CD137L and CD80, wherein the aAPC is capable of inducing proliferation and/or expanding an immune cell population when contacted with the aAPC.

The aAPC may be engineered to express one or more adhesion molecules such as ICAM1/CD54 and/or LFA3/CD58.

In one embodiment, the aAPC comprises a recombinant nucleic acid encoding at least one immune stimulatory ligand and co-stimulatory ligands comprising or consisting of CD86, CD70 and CD137L.

In one embodiment, the aAPC comprises a recombinant nucleic acid encoding at least one immune stimulatory ligand and co-stimulatory ligands comprising or consisting of CD86, CD70, CD137L and CD80. The recombinant nucleic acid may be one that does not occur naturally within the aAPC.

The recombinant nucleic acid may further encode one or more adhesion molecules such as ICAM1/CD54 and/or LFA3/CD58.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e.: rRNA, tRNA and mRNA) or a defined sequence of amino acids and the biological properties resulting therefrom. Thus, a gene encodes a protein if transcription of a gene and translation of mRNA corresponding to that gene produces the protein in a cell or other biological system. Both the coding strand and the non-coding strand, used as the template for transcription of a gene or cDNA, can be referred to as encoding the protein or other product of that gene or cDNA.

In one embodiment, the recombinant nucleic acid further encodes an antigen. In one embodiment, the antigen is a shared antigen that is shared across a patient sub-group.

The patient sub-group may be a sub-group of cancer patients. In one embodiment, the antigen is a shared splice variant antigen. In one embodiment, the antigen is a shared splice variant tumour antigen.

In one embodiment, the recombinant nucleic acid further encodes an antigen such that the antigen is constitutively presented by the immune stimulatory ligand.

In one embodiment the aAPC is capable of expressing and/or presenting an antigen on the surface of the aAPC.

The shared splice variant antigen may be a MAP/microtubule affinity-regulating kinase 3 (MARK3), Neuroblastoma Breakpoint Family Member 9 (NBPF9), Par-3 Family Cell Polarity Regulator (PARD3), Zinc Finger CCCH-Type Containing, Antiviral 1 (ZC3HAV1), YY1 Associated Factor 2 (YAF2), Calcium/calmodulin-dependent protein kinase kinase 1(CAMKK1), Leucine-rich repeat protein 1 (LRR1), Zinc Finger Protein 670 (ZNF670), Glutamate Ionotropic Receptor NMDA Type Subunit Associated Protein 1 (GRINA) or Myeloid Zinc Finger 1 (MZF1) splice variant antigen.

In one embodiment, the MARK3 splice variant antigen comprises a peptide having the sequence of RNMSFRFIK (SEQ ID NO: 1), or encodes a peptide having the sequence of RNMSFRFIK (SEQ ID NO: 1).

In one embodiment, the NBPF9 splice variant antigen comprises a peptide having the sequence of SSFYALEEK (SEQ ID NO: 2), or encodes a peptide having the sequence of SSFYALEEK (SEQ ID NO: 2).

In one embodiment, the PARD3 splice variant antigen comprises a peptide having the sequence of SQLDFVKTRK (SEQ ID NO: 3), or encodes a peptide having the sequence of SQLDFVKTRK (SEQ ID NO: 3).

In one embodiment, the ZC3HAV1 splice variant antigen comprises a peptide having the sequence of LTMAVKAEK (SEQ ID NO: 4), or encodes a peptide having the sequence of LTMAVKAEK (SEQ ID NO: 4).

In one embodiment, the YAF2 splice variant antigen comprises a peptide having the sequence of VIVSASRTK (SEQ ID NO: 5), or encodes a peptide having the sequence of VIVSASRTK (SEQ ID NO: 5).

In one embodiment, the CAMKK1 splice variant antigen comprises a peptide having the sequence of VTSPSRRSK (SEQ ID NO: 6), or encodes a peptide having the sequence of VTSPSRRSK (SEQ ID NO: 6).

In one embodiment, the LRR1 splice variant antigen comprises a peptide having the sequence of SLPRFGYRK (SEQ ID NO: 7), or encodes a peptide having the sequence of SLPRFGYRK (SEQ ID NO: 7).

In one embodiment, the ZNF670 splice variant antigen comprises a peptide having the sequence of SCVSPSSELK (SEQ ID NO: 8), or encodes a peptide having the sequence of SCVSPSSELK (SEQ ID NO: 8).

In one embodiment, the ZC3HAV1 splice variant antigen comprises a peptide having the sequence of LTMAVKAEK (SEQ ID NO: 9), or encodes a peptide having the sequence of LTMAVKAEK (SEQ ID NO: 9).

In one embodiment, the GRINA splice variant antigen comprises a peptide having the sequence of SIRQAFIRK (SEQ ID NO: 10), or encodes a peptide having the sequence of SIRQAFIRK (SEQ ID NO: 10).

In one embodiment, the MZF1 splice variant antigen comprises a peptide having the sequence of KWPPATETL (SEQ ID NO: 11), or encodes a peptide having the sequence of KWPPATETL (SEQ ID NO: 11).

The term “splice variant” as used herein may refer to different mRNA molecules which are a result of differential splicing from the same initial pre-mRNA sequence transcribed from a locus, based upon the inclusion or exclusion of specific exon or intron sequences from the initial pre-mRNA transcript sequence. Each separate splice variant may correlate to a specific polypeptide, based on the amino acid sequence encoded by the processed mRNA.

The term “splice variant” may also refer to a polypeptide encoded by a splice variant of an mRNA transcribed from a locus (also known as an isoform). A single locus may therefore encode multiple protein (or polypeptide) splice variants (or isoforms).

A splice variant may be a nucleic acid (such as an RNA transcript or mRNA) or a polypeptide. The term splice variant may also refer to a fragment of a splice variant nucleic acid or polypeptide.

In one embodiment, the antigen is a tumour-associated antigen selected from p53, Ras, c-Myc, A-Raf, B-Raf, C-Raf, cyclin-dependent kinases, CTA, NY-ESO-1, LAGE-1, MAGE-A1, MAGE-A3, MAGE-A4, MAGE-A10, CT7, CT10, GAGE, PRAME; BAGE; RAGE, SAGE, HAGE, MPHOSPH1, DEPDC1, IMP3 and MAGE-A, BK T-antigen, MAGE-A2, MAGE-A6, MAGE-A12, MART-1, DAM-6, -10, GAGE-1, -2, -8, GAGE-3, -4, -5, -6, -7B, NA88-A, MC1R, Gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumour antigen (WT1), AFP, β-catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RARa, Tumour-associated calcium signal transducer 1 (TACSTD1) TACSTD2, receptor tyrosine kinases, Epidermal Growth Factor receptor (EGFR), EGFRvIII, platelet derived growth factor receptor (PDGFR), vascular endothelial growth factor receptor (VEGFR), cytoplasmic tyrosine kinases, src-family, syk-ZAP70, integrin-linked kinase (ILK), signal transducers and activators of transcription STAT3, STATS, and STAT6, hypoxia inducible factors, HIF-1a and HIF-2a, Nuclear Factor-Kappa B (NF-κB), Notch receptors, Notch1-4, c-Met, mammalian targets of rapamycin (mTOR), WNT, extracellular signal-regulated kinases (ERKs), PMSA, PR-3, MDM2, Mesothelin, renal cell carcinoma—5T4, SM22-alpha, carbonic anhydrases I (CAI) and IX (CAIX), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGs5, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, and fos related antigen 1.

In one embodiment, the antigen is a viral, bacterial or fungal antigen.

Examples of viruses include, but are not limited to, Retroviridae (e.g.: human immunodeficiency viruses, such as HIV-1 (also referred to as HTLV-III, LAV or HTLV-III/LAV, or HIV-III); and other isolates, such as HIV-LP; Picornaviridae (e.g.: polio viruses, hepatitis A virus; enteroviruses, human coxsackie viruses, rhinoviruses, echoviruses); Calciviridae (e.g.: strains that cause gastroenteritis); Togaviridae (e.g.: equine encephalitis viruses, rubella viruses); Flaviridae (e.g.: dengue viruses, encephalitis viruses, yellow fever viruses); Coronaviridae (e.g.: coronaviruses, including SARS-CoV-2 virus); Rhabdoviridae (e.g.: vesicular stomatitis viruses, rabies viruses); Filoviridae (e.g.: ebola viruses); Paramyxoviridae e.g.: parainfluenza viruses, mumps virus, measles virus, respiratory syncytial virus); Orthomyxoviridae e.g.: influenza viruses); Bungaviridae (e.g.: Hantaan viruses, bunga viruses, phleboviruses and Nairo viruses); Arena viridae (hemorrhagic fever viruses); Reoviridae (e.g.: reoviruses, orbiviurses and rotaviruses); Bimaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses); Poxyiridae (variola viruses, vaccinia viruses, pox viruses); and Iridoviridae (e.g.: African swine fever virus); Hepatitis C virus; and unclassified viruses (e.g.: the agent of delta hepatitis (thought to be a defective satellite of hepatitis B virus); Norwalk and related viruses, and astroviruses).

Examples of bacteria include, but are not limited to, Helicobacter pyloris, Borelia burgdorferi, Legionella pneumophila, Mycobacteria sps (e.g.: M. tuberculosis, M. avium, M. intracellulare, M. kansaii, M. gordonae), Staphylococcus aureus, Neisseria gonorrhoeae, Neisseria meningitidis, Listeria monocytogenes, Streptococcus pyrogenes (Group A Streptococcus), Streptococcus agalactiae (Group B Streptococcus), Streptococcus (viridans group), Streptococcus faecalis, Streptococcus bovis, Streptococcus (anaerobic sps.), Streptococcus pneumoniae, pathogenic Campylobacter sp., Enterococcus sp., Haemophilus influenzae, Bacillus anthracis, Corynebacterium diphtheriae, Corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, pathogenic strains of Escherichia coli, Streptobacillus monilformis, Treponema pallidium, Treponema pertenue, Leptospira, and Actinomyces israelli.

Examples of fungi include, but are not limited to, Cryptococcus neoformans, Histoplasma capsulatum, Coccidioides immitis, Blastomyces dermatitidis, Chlamydia trachomatis and Candida albicans.

In one embodiment, the antigen is a viral and/or tumour antigen. The antigen may be EBV F12: AVFDRKSDAK (SEQ ID NO: 12), EBV F28: IVTDFSVIK (SEQ ID NO: 13), NLVPMVATV (SEQ ID NO: 38) or FLLDGSANV (SEQ ID NO: 39).

In one embodiment, the recombinant nucleic acid encodes a fusion protein comprising an antigen and an immune stimulatory ligand. The fusion protein may further comprise a β2-microglobulin polypeptide. The β2-microglobulin polypeptide may be positioned between the antigen and the immune stimulatory ligand.

In one embodiment, the recombinant nucleic acid encodes a fusion protein comprising, in amino-to-carboxy terminal order, an antigen, a first linker, a β2-microglobulin polypeptide, a second linker and an immune stimulatory ligand.

The first linker and the second linker may each be a flexible linker. The term “flexible linker” as used herein refers to a protein molecule containing at least one amino acid residue, usually at least two amino acids residues joined by peptide bond(s), which molecule permits two polypeptides linked thereby to move more freely relative to one another, as compared to their movement without the flexible linker. In certain embodiments, the flexible linker provides increased rotational freedom for two polypeptides linked thereby than the two linked polypeptides would have in the absence of the flexible linker. Such freedom of relative movement or rotational freedom allows polypeptides joined by the flexible linker to perform their individual functions or elicit their activities with less structural hindrance. A flexible linker may be characterized by the absence of secondary structures such as helices or β-sheets or a maximal secondary structure content of 10%, 20% 30% or 40%. Non-limiting examples of flexible linkers include the amino acid sequences GS, GSG, GGSGG, GGSG, GSGS, AS, GGGS, G4S, (G4S)2, (G4S)3, (G4S)4, G4SG, GSGG and GSGGS. Additional flexible linker sequences are well known in the art. In various embodiments, the flexible linker contains or consists of at least 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95 or 100 amino acid residues. In certain embodiments, the flexible linker contains or consists of 1, 2, 3, 4, 5, 6, 7, 8, 9 or 10 amino acid residues.

In one embodiment, the fusion protein comprises an antigen (such as an antigen of any one of SEQ ID NOs: 1-13, 38 and 39). The fusion protein may comprise a first linker of SEQ ID NO: 20. The fusion protein may comprise a β2-microglobulin polypeptide of SEQ ID NO: 21. The fusion protein may comprise a second linker of SEQ ID NO: 22.

The fusion protein may further comprise an immune stimulatory ligand of SEQ ID NO: 23 or 35. The fusion protein may optionally comprise a leader sequence of SEQ ID NO: 19.

In one embodiment, the fusion protein comprises an amino acid sequence of SEQ ID NO: 18.

In one embodiment, there is provided an artificial antigen-presenting cell (aAPC) comprising at least one immune stimulatory ligand, wherein the immune stimulatory ligand is expressed as a fusion protein with an antigen such that the antigen is constitutively presented on the surface of the aAPC. The aAPC may further comprise co-stimulatory ligands comprising or consisting of CD86, CD70 and CD137L. The immune stimulatory ligand may be modified to have attenuated binding affinity to CD8.

In one embodiment, the aAPC is a cell that is transduced with one or more vectors comprising a recombinant nucleic acid encoding at least one immune stimulatory ligand and co-stimulatory ligands comprising CD86, CD70 and CD137L.

In one embodiment, the aAPC is a cell that is transduced with one or more vectors comprising a recombinant nucleic acid encoding at least one immune stimulatory ligand and co-stimulatory ligands comprising CD86, CD70, CD137L and CD80.

In one embodiment, the one or more vectors comprises a recombinant nucleic acid further encoding an antigen.

The recombinant nucleic acid may encode one or more adhesion molecules such as ICAM1/CD54 and/or LFA3/CD58.

In one embodiment, there is provided an artificial antigen-presenting cell (aAPC) comprising at least one immune stimulatory ligand, wherein the immune stimulatory ligand is modified to have attenuated binding affinity to CD8. The immune stimulatory ligand may be modified to have attenuated binding affinity to CD8 as compared to an unmodified immune stimulatory ligand. The attenuated (or weaker) binding affinity may be, for example, a 2, 3, 4, 5, 6, 7, 8, 9, 10, 20, 30, 40, 50, 100 fold or more decrease in binding affinity to CD8 as compared to an unmodified immune stimulatory ligand.

In one embodiment, the immune stimulatory ligand is a major histocompatibility complex polypeptide. In one embodiment, the MHC polypeptide is a MHC Class I or MHC Class II polypeptide. In one embodiment, the MHC class1 polypeptide is HLA-A11. In one embodiment the MHC class 1 polypeptide is HLA-A02. In one embodiment, the MHC class 1 polypeptide is HLA-A24. The MHC class I polypeptide may be loaded with an antigen.

In one embodiment, mutations or substitutions may be introduced in HLA on the aAPC. Such mutations or substitutions may result in further augmentation of the repertoire of TCR sequences recognizing the antigen. For example, mutation of CD8-binding sites on HLA might result in lower-affinity TCR sequences failing to bind to the peptide/HLA complex as their binding is dependent on CD8. This may result in the preferential expansion of T cells that have higher affinity for the HLA/antigen complex.

In one embodiment, the HLA molecule comprises one or more mutations or substitutions that decrease binding affinity of HLA to CD8. The one or more mutations or substitutions may include a mutation or substitution at position 227 (such as D227K), position 228 (such as T228A) or position 245 (such as A245V) in the alpha-3 immunoglobulin domain of HLA. These positions correspond to positions 251, 252 and 270 on SEQ ID NO: 17 respectively. This may generate T cells with a less-differentiated phenotype that may persist better in vivo.

In one embodiment, the stimulatory ligand is an antigen that is recognised by a chimeric antigen receptor (CAR) that is present on a chimeric antigen receptor (CAR) T cell.

The term “Chimeric Antigen Receptor” or “CAR” may refer to a set of polypeptides, which when in an immune effector cell, provide the cell with specificity for a target cell, typically a cancer cell, and with intracellular signal generation. A CAR may comprise at least an extracellular antigen binding domain, a transmembrane domain and a cytoplasmic signaling domain comprising a functional signaling domain derived from a primary signaling domain and/or co-stimulatory domain. The set of polypeptides may be contiguous with each other. In some embodiments, the set of polypeptides includes a dimerization switch that, upon the presence of a dimerization molecule, can couple the polypeptides to one another, e.g.: can couple an antigen binding domain to an intracellular signaling domain.

In one embodiment, the aAPC encodes a single immune stimulatory ligand (i.e.: only one MHC polypeptide). This is so that the aAPC can be caused to present only the antigen or antigens provided to it.

In one embodiment, the aAPC is a recombinant cell. The recombinant cell may be a recombinant immune cell or a recombinant non-immune cell.

The term “recombinant” includes reference to a cell that has been modified by the introduction of a heterologous nucleic acid, or a cell derived from a cell that has been modified in such a manner, but does not encompass the alteration of the cell by naturally occurring events (e.g.: spontaneous mutation, natural transformation, natural transduction, natural transposition) such as those occurring without deliberate human intervention. The recombinant cell may be a non-naturally occurring cell. The recombinant cell may also be an engineered cell.

In one embodiment, the aAPC is a mammalian cell. The mammalian cell may be a live mammalian cell. In one embodiment, the aAPC is a non-immune cell. In one embodiment, the aAPC is a tumour or fibroblast cell.

In one embodiment, the aAPC is a myeloid cell. In one embodiment, the aAPC is a K562 cell. In one embodiment, the aAPC is a HEK293T cell. In one embodiment, the aAPC is T2 cell.

The term “immune cell” as referred to herein includes cells that are of haematopoietic origin and that play a role in the immune response. Immune cells include lymphocytes, such as B cells and T cells; natural killer (NK) cells; myeloid cells, such as monocytes, macrophages, dendritic cells, eosinophils, mast cells, basophils, and granulocytes.

In one embodiment, the immune cell is a T cell or an NK cell. In one embodiment, the immune cell is a T cell.

In one embodiment, the immune cell is a chimeric antigen receptor (CAR)-expressing immune cell (such as a CAR T cell or CAR NK cell).

In one embodiment, the immune cell is a T cell receptor (TCR)-expressing immune cell (such as a TCR-engineered T cell).

The term “T cell” may refer to a CD4+ T cell (such as an immature CD4+ T cell or a mature CD4+ helper T cell). The term “T cell” may also refer to a CD8+ T cell (such as an immature CD8+ T cell or a mature CD8+ cytotoxic T cell). The term “T cells” may also refer to a mixture of CD4+ T cells as well as CD8+ T cells.

In some embodiments, the T cell is a non-naïve T cell. In some embodiments, the T cell is a naïve T cell. In some embodiments, the T cell might also refer to an antigen-experienced T cell.

In some embodiments, the T lymphocyte is a cytotoxic T cell. A cytotoxic T cell (also known as cytotoxic T lymphocyte, Tc, CTL, T-killer cell, cytolytic T cell, CD8+ T cell or killer T cell) is a T cell that kills cancer cells, infected cells or cells that are damaged in other ways.

In some embodiments, the T cell is a helper T cell. A helper T cell is a T cell that helps the activity of other immune cells by releasing T cell cytokines to regulate immune responses.

Isolated Nucleic Acid/Vector

Disclosed herein is an isolated nucleic acid encoding at least one immune stimulatory ligand and co-stimulatory ligands comprising or consisting of CD86, CD70 and CD137L.

Disclosed herein is an isolated nucleic acid encoding at least one immune stimulatory ligand and an isolated nucleic acid encoding co-stimulatory ligands comprising or consisting of CD86, CD70 and CD137L.

Disclosed herein is an isolated nucleic acid encoding at least one immune stimulatory ligand. In one embodiment, the isolated nucleic acid encodes a fusion protein comprising an antigen and an immune stimulatory ligand. The isolated nucleic acid may further encode a β2-microglobulin polypeptide positioned between the antigen and the immune stimulatory ligand in the fusion protein.

Disclosed herein is an isolated nucleic acid encoding a fusion protein comprising, in amino-to-carboxy terminal order, an antigen, a first linker, a β2-microglobulin polypeptide, a second linker and an immune stimulatory ligand.

Disclosed herein is an isolated nucleic acid encoding a) a fusion protein comprising, in amino-to-carboxy terminal order, an antigen, a first linker, a β2-microglobulin polypeptide, a second linker and an immune stimulatory ligand, and b) co-stimulatory ligands comprising or consisting of CD86, CD70 and CD137L.

The term “polynucleotide” or “nucleic acid” as used herein designates mRNA, RNA, cDNA or DNA. The term typically refers to polymeric forms of nucleotides of at least 10 bases in length, either ribonucleotides or deoxyribonucleotides or a modified form of either type of nucleotide. The term includes single and double stranded forms of DNA.

An “isolated nucleic acid” refers to a nucleic acid segment or fragment which has been separated from sequences which flank it in a naturally occurring state, e.g.: a DNA fragment which has been removed from the sequences which are normally adjacent to the fragment, e.g.: the sequences adjacent to the fragment in a genome in which it naturally occurs. The term also applies to nucleic acids which have been substantially purified from other components which naturally accompany the nucleic acid, e.g.: RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g.: as a cDNA or a genomic or cDNA fragment produced by PCR or restriction enzyme digestion) independent of other sequences. It also includes a recombinant DNA which is part of a hybrid gene encoding additional polypeptide sequence.

In one embodiment, the isolated nucleic acid is operably linked to one or more expression control sequences.

The term “operably linked” as used herein refers to a juxtaposition wherein the components so described are in a relationship permitting them to function in their intended manner. For example, a regulatory sequence (e.g.: a promoter) “operably linked” to a nucleotide sequence of interest (e.g.: a coding and/or non-coding sequence) refers to positioning and/or orientation of the control sequence relative to the nucleotide sequence of interest to permit expression of that sequence under conditions compatible with the control sequence. The control sequences need not be contiguous with the nucleotide sequence of interest, so long as they function to direct its expression. Thus, for example, intervening non-coding sequences (e.g.: untranslated, yet transcribed, sequences) can be present between a promoter and a coding sequence, and the promoter sequence can still be considered “operably linked” to the coding sequence.

Disclosed herein are one or more vectors comprising an isolated nucleic acid as defined herein. In one embodiment, the vector is a viral vector. The viral vector may be a lentiviral vector. In one embodiment, the vector is an expression vector.

By the term “vector”, as used herein, is meant any plasmid or virus encoding an exogenous nucleic acid. The term should also be construed to include non-plasmid and non-viral compounds which facilitate transfer of nucleic acid into virions or cells, such as, for example, polylysine compounds and the like. The vector may be a viral vector which is suitable as a delivery vehicle for delivery of a nucleic acid that encodes a protein and/or antibody of the invention, to the patient, or to the aAPC, or the vector may be a non-viral vector which is suitable for the same purpose. Examples of viral and non-viral vectors for delivery of DNA to cells and tissues are well known in the art and are described, for example, in Ma et al. (1997, Proc. Natl. Acad. Sci. U.S.A. 94: 12744-12746). Examples of viral vectors include, but are not limited to, a lentiviral vector, a recombinant adenovirus, a recombinant retrovirus, a recombinant adeno-associated virus, a recombinant avian pox virus, and the like (Cranage et al., 1986, EMBO J. 5:3057-3063; International Patent Application No. WO94/17810, published Aug. 18, 1994; International Patent Application No. WO94/23744, published Oct. 27, 1994).

Examples of non-viral vectors include, but are not limited to, liposomes, polyamine derivatives of DNA, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g.: naked or contained in liposomes) and viruses (e.g.: retroviruses, lentiviruses, adenoviruses, and adeno-associated viruses) that incorporate the recombinant polynucleotide.

Polypeptide/Compositions

Provided herein is a polypeptide or polypeptides encoded by an isolated nucleic acid as defined herein.

Provided herein is a composition (such as a vaccine) comprising a polypeptide or polypeptides as defined herein.

Methods of Preparing an aAPC and/or Generating/Expanding Immune Cells

Disclosed herein is a method of preparing an aAPC as defined herein, wherein the method comprises transfecting or transducing a mammalian cell with one or more isolated nucleic acids or one or more vectors as defined herein.

In one embodiment, the mammalian cell is transiently transfected or transduced with one or more isolated nucleic acids or one or more vectors as defined herein.

In one embodiment, the mammalian cell is stably transfected or transduced with one or more isolated nucleic acids or one or more vectors as defined herein.

Disclosed herein is a method of detecting or identifying a population of antigen-specific immune cells, said method comprising contacting a population of immune cells with an aAPC as defined herein to detect or identify a population of antigen-specific immune cells.

Disclosed herein is a method for inducing proliferation of an antigen-specific immune cell, said method comprising contacting said antigen-specific immune cell with an aAPC as defined herein, thereby inducing proliferation of the antigen-specific immune cell.

Disclosed herein is a method for expanding a population of antigen-specific immune cells, said method comprising contacting the population of antigen-specific immune cells with an aAPC as defined herein, thereby expanding the population of the antigen-specific immune cells.

In one embodiment, the method comprises contacting the population of antigen-specific immune cells with an aAPC comprising at least one immune stimulatory ligand, wherein the immune stimulatory ligand is modified to have attenuated binding affinity to CD8, thereby expanding the population of the antigen-specific immune cells.

In one embodiment, the modified immune stimulatory ligand is a MHC Class I or MHC Class II polypeptide. The MHC class1 polypeptide may be any one of HLA-A11, HLA-A02 or HLA-A24. In one embodiment, the HLA polypeptide is loaded with an antigen.

This may result in the preferential expansion of a population of immune cells having higher affinity for the HLA/antigen complex.

In one embodiment, the method comprises contacting a population of immune cells with two or more distinct aAPCs as defined herein to expand two or more distinct populations of antigen-specific immune cells.

Disclosed herein is a method of generating a population of antigen-specific immune cells, said method comprising contacting a population of immune cells with an aAPC as defined herein to generate a population of antigen-specific immune cells.

In one embodiment, the method comprises contacting the population of antigen-specific immune cells with an aAPC comprising at least one immune stimulatory ligand, wherein the immune stimulatory ligand is modified to have attenuated binding affinity to CD8, thereby expanding the population of the antigen-specific immune cells.

In one embodiment, the modified immune stimulatory ligand is a MHC Class I or MHC Class II polypeptide. The MHC class1 polypeptide may be any one of HLA-A11, HLA-A02 or HLA-A24. In one embodiment, the HLA polypeptide is loaded with an antigen.

This may result in the preferential expansion of a population of immune cells having higher affinity for the HLA/antigen complex.

In one embodiment, the method comprises contacting a population of immune cells with two or more distinct aAPCs as defined herein to generate two or more distinct populations of antigen-specific immune cells.

The immune cell as referred to herein may be a T cell, such as a naïve T cell, a memory T cell or a TCR engineered T cell. The immune cell may be derived from healthy subjects or may be derived from patients suffering from a medical condition such as, for example, cancer. The aAPC may be loaded with an antigen. The antigen may, for example, be presented on the surface of the aAPC as a fusion protein with an MHC molecule. The antigen can for, example, be a spliced variant antigen or a tumour-associated antigen as defined herein.

In one embodiment, the immune cell may be from a HLA-mismatched donor. By HLA-mismatched donor, it is meant that the immune cell (such as a T cell) is obtained from a subject having different HLA molecules from the HLA molecule present in the aAPC.

This allows a greater or different repertoire of antigen-specific immune cells to be generated due to differences in TCR repertoire in these HLA-mismatched individuals.

The step of contacting the antigen-specific immune cell or population of antigen-specific immune cells with an aAPC may result in the proliferation of antigen-specific immune cells that are antigen-specific towards the antigen that is presented on the surface of the aAPC. These immune cells can be isolated to obtain the sequences of the corresponding immune stimulatory molecules (e.g.: TCR sequences). Alternatively, the immune cells can be used to treat a medical condition in a subject.

The method as referred to herein may further comprise contacting a population of immune cells with two or more aAPCs, each presenting a distinct antigen, resulting in expansion of two or more populations of antigen-specific immune cells.

Treatment

In one embodiment, there is provided a pharmaceutical composition comprising an aAPC as defined herein. The pharmaceutical composition may comprise a pharmaceutically acceptable carrier.

By “pharmaceutically acceptable carrier” is meant a solid or liquid filler, diluent or encapsulating substance that can be used safely in topical or systemic administration to an animal, preferably a mammal, including humans. Representative pharmaceutically acceptable carriers include any and all solvents, dispersion media, coatings, surfactants, antioxidants, preservatives (e.g.: antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference).

Except insofar as any conventional carrier is incompatible with the active ingredient(s), its use in the pharmaceutical compositions is contemplated.

Disclosed herein is an aAPC as defined herein for use as a medicament.

Disclosed herein is a method for inducing an immune cell response to an antigen in a subject, the method comprising administering the aAPC as defined herein to the subject, wherein the aAPC comprises an MHC Class I molecule loaded with the antigen, wherein the aAPC induces proliferation of an immune cell specific for the antigen, thereby inducing an immune cell response to the antigen in the subject.

Disclosed herein is a method for inducing an immune cell response to an antigen in a subject, the method comprising administering the aAPC as defined herein to the subject, wherein the aAPC induces proliferation of an immune cell specific for the antigen, thereby inducing an immune cell response to the antigen in the subject.

In one embodiment, there is provided an aAPC as defined herein for use in inducing an immune cell response to an antigen in a subject.

In one embodiment, there is provided use of an aAPC as defined herein in the manufacture of a medicament for inducing an immune cell response to an antigen in a subject.

The term “administering” refers to contacting, applying or providing a suitable therapy to a subject suffering from a medical condition. The medical condition may be cancer and the suitable therapy may be any one of a number of anti-cancer immunotherapies.

The terms “patient”, “subject”, “host” or “individual”, used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the phylum Chordata including primates (e.g.: humans, monkeys and apes, and includes species of monkeys such from the genus Macaca (e.g.: cynomologus monkeys such as Macaca fascicularis, and/or rhesus monkeys (Macaca mulatta) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g.: mice rats, guinea pigs), lagomorphs (e.g.: rabbits, hares), bovines (e.g.: cattle), ovines (e.g.: sheep), caprines (e.g.: goats), porcines (e.g.: pigs), equines (e.g.: horses), canines (e.g.: dogs), felines (e.g.: cats), avians (e.g.: chickens, turkeys, ducks, geese, companion birds such as canaries, budgerigars etc.), marine mammals (e.g.: dolphins, whales), reptiles (snakes, frogs, lizards etc.), and fish. In some embodiments, the subject is human.

Disclosed herein is a method of treating a medical condition in a subject, the method comprising administering a population of antigen-specific immune cells as defined herein to the subject to treat the medical condition in the subject.

In one embodiment, there is provided an antigen-specific population of immune cells as defined herein for use in treating a medical condition in a subject.

In one embodiment, there is provided the use of an antigen-specific population of immune cells as defined herein in the manufacture of a medicament for treating a medical condition in a subject.

In one embodiment, there is provided a method of treating a medical condition in a subject, the method comprising: a) isolating a population of immune cells that binds specifically to an antigen associated with the medical condition; b) expanding the population of immune cells by contacting the population of immune cells with an aAPC as defined herein; and c) administering the expanded population of immune cells to the subject to treat the medical condition in the subject.

Disclosed herein is a method of treating a medical condition in a subject, the method comprising: a) expanding a population of immune cells that has been isolated from the subject by contacting the population of immune cells with an aAPC as defined herein; and b) administering the expanded population of immune cells to the subject to treat the medical condition in the subject.

The term “treating” as used herein may refer to (1) preventing or delaying the appearance of one or more symptoms of the disorder; (2) inhibiting the development of the disorder or one or more symptoms of the disorder; (3) relieving the disorder, i.e.: causing regression of the disorder or at least one or more symptoms of the disorder; and/or (4) causing a decrease in the severity of one or more symptoms of the disorder.

In one embodiment, the medical condition as referred to herein is a cancer. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. By “non-metastatic” is meant a cancer that remains at the primary site and has not penetrated into the lymphatic or blood vessel system or to tissues other than the primary site. The term “metastatic cancer” refers to cancer that has spread or is capable of spreading from one part of the body to another. Generally, a non-metastatic cancer is any cancer that is a Stage 0, I, or II cancer, and occasionally a Stage III cancer. A metastatic cancer, on the other hand, is usually a stage IV cancer.

The term “cancer” includes but is not limited to, breast cancer, large intestinal cancer, lung cancer, small cell lung cancer, gastric (stomach) cancer, liver cancer, blood cancer, bone cancer, pancreatic cancer, skin cancer, head and/or neck cancer, cutaneous or intraocular melanoma, uterine sarcoma, ovarian cancer, rectal or colorectal cancer, anal cancer, colon cancer, fallopian tube carcinoma, endometrial carcinoma, cervical cancer, vulval cancer, squamous cell carcinoma, vaginal carcinoma, Hodgkin's disease, non-Hodgkin's lymphoma, oesophageal cancer, small intestine cancer, endocrine cancer, thyroid cancer, parathyroid cancer, adrenal cancer, soft tissue tumour, urethral cancer, penile cancer, prostate cancer, chronic or acute leukaemia, lymphocytic lymphoma, bladder cancer, kidney cancer, ureter cancer, renal cell carcinoma, renal pelvic carcinoma, CNS tumour, glioma, astrocytoma, glioblastoma multiforme, primary CNS lymphoma, bone marrow tumour, brain stem nerve gliomas, pituitary adenoma, uveal melanoma (also known as intraocular melanoma), testicular cancer, oral cancer, pharyngeal cancer or a combination thereof.

In some embodiments, the cancer is gastric cancer, head and neck cancer, colorectal cancer or hepatocellular cancer. In some embodiments, the cancer is gastric cancer or colorectal cancer.

In some embodiments, the cancer is gastric cancer. In some embodiments, the cancer is head and/or neck cancer. In some embodiments, the cancer is colorectal cancer. In some embodiments, the cancer is hepatocellular cancer. In some embodiments, the cancer is breast cancer.

In some embodiments, the cancer is one that is characterised by the expression of one or more shared antigens. The cancer may be found in any location of the body, but is defined by the expression of the one or more shared antigens.

In some embodiments, the cancer is a metastatic cancer. The metastatic cancer may be found in different locations of the body but is characterised by the expression of the one or more shared antigens.

In one embodiment, the medical condition as referred to herein is a viral infection. The viral infection may be an infection by a pathogenic virus. Pathogenic viruses may include, but are not limited to, Retroviridae (e.g.: human immunodeficiency viruses, such as HIV-1 (also referred to as HDTV-III, LAVE or HTLV-III/LAV, or HIV-III; and other isolates, such as HIV-LP; Picornaviridae (e.g.: polio viruses, hepatitis A virus; enteroviruses, human Coxsackie viruses, rhinoviruses, echoviruses); Calciviridae (e.g.: strains that cause gastroenteritis); Togaviridae (e.g.: equine encephalitis viruses, rubella viruses); Flaviridae (e.g.: dengue viruses, encephalitis viruses, yellow fever viruses); Coronoviridae (e.g.: coronaviruses (including SARS-CoV or SARS-CoV-2); Rhabdoviradae e.g.: vesicular stomatitis viruses, rabies viruses); Filoviridae (e.g.: ebola viruses); Paramyxoviridae (e.g.: parainfluenza viruses, mumps virus, measles virus, respiratory syncytial virus); Orthomyxoviridae e.g.: influenza viruses); Bungaviridae (e.g.: Hantaan viruses, bunga viruses, phleboviruses and Nairo viruses); Arena viridae (hemorrhagic fever viruses); Reoviridae (e.g.: reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes virus; Poxviridae (variola viruses, vaccinia viruses, pox viruses); and Iridoviridae (e.g.: African swine fever virus).

The methods as defined herein may comprise administering an effective amount of the aAPCs or antigen-specific immune cells as defined herein to the subject.

By “effective amount”, in the context of treating, inhibiting the development of, or preventing a condition is meant the administration of an amount of an agent or composition to an individual in need of such treatment, inhibition or prophylaxis, either in a single dose or as part of a series, that is effective for the prevention of incurring a symptom, holding in check such symptoms, and/or treating existing symptoms, of that condition. The effective amount will vary depending upon the health and physical condition of the individual to be treated, the taxonomic group of individual to be treated, the formulation of the composition, the assessment of the medical situation, and other relevant factors. It is expected that the amount will fall in a relatively broad range that can be determined through routine trials.

Screening

Disclosed herein is a method of identifying an antigenic peptide in a subject, the method comprising: a) contacting an aAPC as defined herein with a population of immune cells that has been obtained from the subject, wherein the aAPC is loaded with a candidate peptide or comprises a recombinant nucleic acid encoding a candidate peptide; and b) detecting a population of immune cells that recognize the candidate peptide, thus identifying the candidate peptide as an antigenic peptide.

Disclosed herein is a method for identifying or detecting the presence of an immune cell that recognizes a desired antigen, said method comprising contacting said immune cell with one or more aAPCs as disclosed herein that presents said desired antigens, and identifying or detecting the presence of a population of immune cells that recognize said one or more desired antigens.

In one embodiment, the population of immune cells that recognize the candidate peptide is detected with an ELISPOT assay (such as an IFN-γ ELISPOT assay) and/or enumeration of antigen-specific T cells.

The method may further comprise using the aAPC for further expansion of antigen-specific immune cells. The expanded population of antigen-specific immune cells may be used for the treatment of a subject or for isolation of TCR sequences.

The articles “a” and “an” are used herein to refer to one or to more than one (i.e.: to at least one) of the grammatical object of the article. By way of example, “a vector” means one vector or more than one vector.

Throughout this specification and the statements which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

Those skilled in the art will appreciate that the invention described herein in susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features. Certain embodiments of the invention will now be described with reference to the following examples which are intended for the purpose of illustration only and are not intended to limit the scope of the generality hereinbefore described.

### EXAMPLES

Materials

K562 Artificial Antigen-Presenting Cells (aAPC)

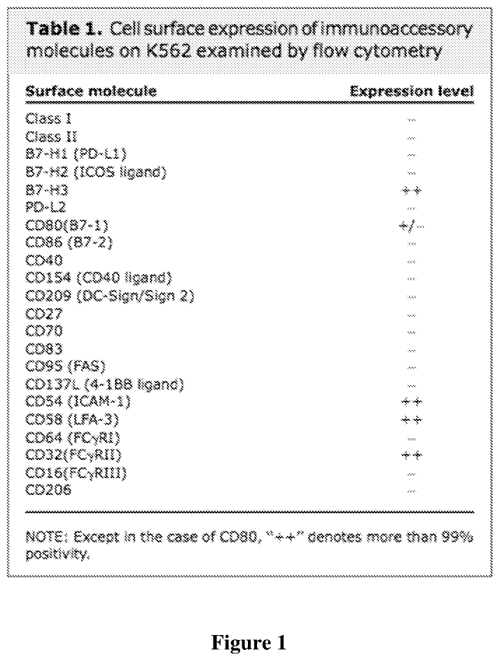

A lentiviral construct containing HLA-A11 and ligands for co-stimulatory molecules (CD86, CD70 and CD137L) was made (FIG. 3). Lentiviral particles were made and used to transduce the erythroleukemia K562 cell line. K562 cell lines do not express HLA molecules but endogenously express some T cell co-stimulatory ligands (e.g. CD80) (see Table 1 in FIG. 1). Stable cell lines expressing HLA-A11 and co-stimulatory ligands were obtained by selection with antibiotics and sorting for surface expression of the HLA-A11 and co-stimulatory ligands.

The amino acid sequence of CD86 (Uniprot: P42081) is provided below:

(SEQ ID NO: 14)
MDPQCTMGLSNILFVMAFLLSGAAPLKIQAYFNETADLPCQFANSQNQS
LSELVVFWQDQENLVLNEVYLGKEKFDSVHSKYMGRTSFDSDSWTLRLH
NLQIKDKGLYQCIIHHKKPTGMIRIHQMNSELSVLANFSQPEIVPISNI
TENVYINLTCSSIHGYPEPKKMSVLLRTKNSTIEYDGIMQKSQDNVTEL
YDVSISLSVSFPDVTSNMTIFCILETDKTRLLSSPFSIELEDPQPPPDH
IPWITAVLPTVIICVMVFCLILWKWKKKKRPRNSYKCGTNTMEREESEQ
TKKREKIHIPERSDEAQRVFKSSKTSSCDKSDTCF

The amino acid sequence of CD70 (Uniprot: P32970) is provided below:

(SEQ ID NO: 15)
MPEEGSGCSVRRRPYGCVLRAALVPLVAGLVICLVVCIQRFAQAQQQLP
LESLGWDVAELQLNHTGPQQDPRLYWQGGPALGRSFLHGPELDKGQLRI
HRDGIYMVHIQVTLAICSSTTASRHHPTTLAVGICSPASRSISLLRLSF
HQGCTIASQRLTPLARGDTLCTNLTGTLLPSRNTDETFFGVQWVRP

The amino acid sequence of CD137L (Uniprot: P41273) is provided below:

(SEQ ID NO: 16)
MEYASDASLDPEAPWPPAPRARACRVLPWALVAGLLLLLLLAAACAVFL
ACPWAVSGARASPGSAASPRLREGPELSPDDPAGLLDLRQGMFAQLVAQ
NVLLIDGPLSWYSDPGLAGVSLTGGLSYKEDTKELVVAKAGVYYVFFQL
ELRRVVAGEGSGSVSLALHLQPLRSAAGAAALALTVDLPPASSEARNSA
FGFQGRLLHLSAGQRLGVHLHTEARARHAWQLTQGATVLGLFRVTPEIP
AGLPSPRSE

The amino acid sequence of HLA-A11 (with HLA-A11 leader sequence) is provided below:

(SEQ ID NO: 17)
MAVMAPRTLLLLLSGALALTQTWAGSHSMRYFYTSVSRPGRGEPRFIAV
GYVDDTQFVRFDSDAASQRMEPRAPWIEQEGPEYWDQETRNVKAQSQTD
RVDLGTLRGYYNQSEDGSHTIQIMYGCDVGPDGRFLRGYRQDAYDGKDY
IALNEDLRSWTAADMAAQITKRKWEAAHAAEQQRAYLEGRCVEWLRRYL
ENGKETLQRTDPPKTHMTHHPISDHEATLRCWALGFYPAEITLTWQRDG
EDQTQDTELVETRPAGDGTFQKWAAVVVPSGEEQRYTCHVQHEGLPKPL
TLRWELSSQPTIPIVGIIAGLVLLGAVITGAVVAAVMWRRKSSDRKGGS
YTQAASSDSAQGSDVSLTACKV

Single Chain (SC)-HLA Constructs

MARK3 SC HLA-A11
(SEQ ID NO: 18)
MSRSVALAVLALLSLSGLEARNMSFRFIKGGGGSGGGGSGGGGSIQRTP
KIQVYSRHPAENGKSNFLNCYVSGFHPSDIEVDLLKNGERIEKVEHSDL
SFSKDWSFYLLYYTEFTPTEKDEYACRVNHVTLSQPKIVKWDRDMGGGG
SGGGGSGGGGSGGGGSGSHSMRYFYTSVSRPGRGEPRFIAVGYVDDTQF
VRFDSDAASQRMEPRAPWIEQEGPEYWDQETRNVKAQSQTDRVDLGTLR
GYYNQSEDGSHTIQIMYGCDVGPDGRFLRGYRQDAYDGKDYIALNEDLR
SWTAADMAAQITKRKWEAAHAAEQQRAYLEGRCVEWLRRYLENGKETLQ
RTDPPKTHMTHHPISDHEATLRCWALGFYPAEITLTWQRDGEDQTQDTE
LVETRPAGDGTFQKWAAVVVPSGEEQRYTCHVQHEGLPKPLTLRWELSS
QPTIPIVGIIAGLVLLGAVITGAVVAAVMWRRKSSDRKGGSYTQAASSD
SAQGSDVSLTACKV

The amino acid sequence of the leader Sequence (i.e. a B2M leader Sequence) is provided below:

(SEQ ID NO: 19)
MSRSVALAVLALLSLSGLEA

The amino acid sequence of the GS linker 1 is provided below:

(SEQ ID NO: 20)
GGGGSGGGGSGGGGS

The amino acid sequence of β2-microglobulin (B2M) is provided below:

(SEQ ID NO: 21)
IQRTPKIQVYSRHPAENGKSNFLNCYVSGFHPSDIEVDLLKNGERIEKV
EHSDLSFSKDWSFYLLYYTEFTPTEKDEYACRVNHVTLSQPKIVKWDRD
M

The amino acid sequence of the GS linker 2 is provided below:

(SEQ ID NO: 22)
GGGGSGGGGSGGGGSGGGGS

The amino acid sequence of HLA-A11 is provided below:

(SEQ ID NO: 23)
GSHSMRYFYTSVSRPGRGEPRFIAVGYVDDTQFVRFDSDAASQRMEPRA
PWIEQEGPEYWDQETRNVKAQSQTDRVDLGTLRGYYNQSEDGSHTIQIM
YGCDVGPDGRFLRGYRQDAYDGKDYIALNEDLRSWTAADMAAQITKRKW
EAAHAAEQQRAYLEGRCVEWLRRYLENGKETLQRTDPPKTHMTHHPISD
HEATLRCWALGFYPAEITLTWQRDGEDQTQDTELVETRPAGDGTFQKWA
AVVVPSGEEQRYTCHVQHEGLPKPLTLRWELSSQPTIPIVGIIAGLVLL
GAVITGAVVAAVMWRRKSSDRKGGSYTQAASSDSAQGSDVSLTACKV
NBPF9 SC HLA-A11
(SEQ ID NO: 24)
MSRSVALAVLALLSLSGLEASSFYALEEKGGGGSGGGGSGGGGSIQRTP
KIQVYSRHPAENGKSNFLNCYVSGFHPSDIEVDLLKNGERIEKVEHSDL
SFSKDWSFYLLYYTEFTPTEKDEYACRVNHVTLSQPKIVKWDRDMGGGG
SGGGGSGGGGSGGGGSGSHSMRYFYTSVSRPGRGEPRFIAVGYVDDTQF
VRFDSDAASQRMEPRAPWIEQEGPEYWDQETRNVKAQSQTDRVDLGTLR
GYYNQSEDGSHTIQIMYGCDVGPDGRFLRGYRQDAYDGKDYIALNEDLR
SWTAADMAAQITKRKWEAAHAAEQQRAYLEGRCVEWLRRYLENGKETLQ
RTDPPKTHMTHHPISDHEATLRCWALGFYPAEITLTWQRDGEDQTQDTE
LVETRPAGDGTFQKWAAVVVPSGEEQRYTCHVQHEGLPKPLTLRWELSS
QPTIPIVGIIAGLVLLGAVITGAVVAAVMWRRKSSDRKGGSYTQAASSD
SAQGSDVSLTACKV
PARD3 SC HLA-A11
(SEQ ID NO: 25)
MSRSVALAVLALLSLSGLEASQLDFVKTRKGGGGSGGGGSGGGGSIQRT
PKIQVYSRHPAENGKSNFLNCYVSGFHPSDIEVDLLKNGERIEKVEHSD
LSFSKDWSFYLLYYTEFTPTEKDEYACRVNHVTLSQPKIVKWDRDMGGG
GSGGGGSGGGGSGGGGSGSHSMRYFYTSVSRPGRGEPRFIAVGYVDDTQ
FVRFDSDAASQRMEPRAPWIEQEGPEYWDQETRNVKAQSQTDRVDLGTL
RGYYNQSEDGSHTIQIMYGCDVGPDGRFLRGYRQDAYDGKDYIALNEDL
RSWTAADMAAQITKRKWEAAHAAEQQRAYLEGRCVEWLRRYLENGKETL
QRTDPPKTHMTHHPISDHEATLRCWALGFYPAEITLTWQRDGEDQTQDT
ELVETRPAGDGTFQKWAAVVVPSGEEQRYTCHVQHEGLPKPLTLRWELS
SQPTIPIVGIIAGLVLLGAVITGAVVAAVMWRRKSSDRKGGSYTQAASS
DSAQGSDVSLTACKV
ZC3HAV1 SC HLA-A11
(SEQ ID NO: 26)
MSRSVALAVLALLSLSGLEALTMAVKAEKGGGGSGGGGSGGGGSIQRTP
KIQVYSRHPAENGKSNFLNCYVSGFHPSDIEVDLLKNGERIEKVEHSDL
SFSKDWSFYLLYYTEFTPTEKDEYACRVNHVTLSQPKIVKWDRDMGGGG
SGGGGSGGGGSGGGGSGSHSMRYFYTSVSRPGRGEPRFIAVGYVDDTQF
VRFDSDAASQRMEPRAPWIEQEGPEYWDQETRNVKAQSQTDRVDLGTLR
GYYNQSEDGSHTIQIMYGCDVGPDGRFLRGYRQDAYDGKDYIALNEDLR
SWTAADMAAQITKRKWEAAHAAEQQRAYLEGRCVEWLRRYLENGKETLQ
RTDPPKTHMTHHPISDHEATLRCWALGFYPAEITLTWQRDGEDQTQDTE
LVETRPAGDGTFQKWAAVVVPSGEEQRYTCHVQHEGLPKPLTLRWELSS
QPTIPIVGIIAGLVLLGAVITGAVVAAVMWRRKSSDRKGGSYTQAASSD
SAQGSDVSLTACKV
YAF2 SC HLA-A11
(SEQ ID NO: 27)
MSRSVALAVLALLSLSGLEAVIVSASRTKGGGGSGGGGSGGGGSIQRTP
KIQVYSRHPAENGKSNFLNCYVSGFHPSDIEVDLLKNGERIEKVEHSDL
SFSKDWSFYLLYYTEFTPTEKDEYACRVNHVTLSQPKIVKWDRDMGGGG
SGGGGSGGGGSGGGGSGSHSMRYFYTSVSRPGRGEPRFIAVGYVDDTQF
VRFDSDAASQRMEPRAPWIEQEGPEYWDQETRNVKAQSQTDRVDLGTLR
GYYNQSEDGSHTIQIMYGCDVGPDGRFLRGYRQDAYDGKDYIALNEDLR
SWTAADMAAQITKRKWEAAHAAEQQRAYLEGRCVEWLRRYLENGKETLQ
RTDPPKTHMTHHPISDHEATLRCWALGFYPAEITLTWQRDGEDQTQDTE
LVETRPAGDGTFQKWAAVVVPSGEEQRYTCHVQHEGLPKPLTLRWELSS
QPTIPIVGIIAGLVLLGAVITGAVVAAVMWRRKSSDRKGGSYTQAASSD
SAQGSDVSLTACKV
CAMKK1 SC HLA-A11
(SEQ ID NO: 28)
MSRSVALAVLALLSLSGLEAVTSPSRRSKGGGGSGGGGSGGGGSIQRTP
KIQVYSRHPAENGKSNFLNCYVSGFHPSDIEVDLLKNGERIEKVEHSDL
SFSKDWSFYLLYYTEFTPTEKDEYACRVNHVTLSQPKIVKWDRDMGGGG
SGGGGSGGGGSGGGGSGSHSMRYFYTSVSRPGRGEPRFIAVGYVDDTQF
VRFDSDAASQRMEPRAPWIEQEGPEYWDQETRNVKAQSQTDRVDLGTLR
GYYNQSEDGSHTIQIMYGCDVGPDGRFLRGYRQDAYDGKDYIALNEDLR
SWTAADMAAQITKRKWEAAHAAEQQRAYLEGRCVEWLRRYLENGKETLQ
RTDPPKTHMTHHPISDHEATLRCWALGFYPAEITLTWQRDGEDQTQDTE
LVETRPAGDGTFQKWAAVVVPSGEEQRYTCHVQHEGLPKPLTLRWELSS
QPTIPIVGIIAGLVLLGAVITGAVVAAVMWRRKSSDRKGGSYTQAASSD
SAQGSDVSLTACKV
LRR1 SC HLA-A11
(SEQ ID NO: 29)
MSRSVALAVLALLSLSGLEASLPRFGYRKGGGGSGGGGSGGGGSIQRTP
KIQVYSRHPAENGKSNFLNCYVSGFHPSDIEVDLLKNGERIEKVEHSDL
SFSKDWSFYLLYYTEFTPTEKDEYACRVNHVTLSQPKIVKWDRDMGGGG
SGGGGSGGGGSGGGGSGSHSMRYFYTSVSRPGRGEPRFIAVGYVDDTQF
VRFDSDAASQRMEPRAPWIEQEGPEYWDQETRNVKAQSQTDRVDLGTLR
GYYNQSEDGSHTIQIMYGCDVGPDGRFLRGYRQDAYDGKDYIALNEDLR
SWTAADMAAQITKRKWEAAHAAEQQRAYLEGRCVEWLRRYLENGKETLQ
RTDPPKTHMTHHPISDHEATLRCWALGFYPAEITLTWQRDGEDQTQDTE
LVETRPAGDGTFQKWAAVVVPSGEEQRYTCHVQHEGLPKPLTLRWELSS
QPTIPIVGIIAGLVLLGAVITGAVVAAVMWRRKSSDRKGGSYTQAASSD
SAQGSDVSLTACKV
ZNF670 SC HLA-A11
(SEQ ID NO: 30)
MSRSVALAVLALLSLSGLEASCVSPSSELKGGGGSGGGGSGGGGSIQRT
PKIQVYSRHPAENGKSNFLNCYVSGFHPSDIEVDLLKNGERIEKVEHSD
LSFSKDWSFYLLYYTEFTPTEKDEYACRVNHVTLSQPKIVKWDRDMGGG
GSGGGGSGGGGSGGGGSGSHSMRYFYTSVSRPGRGEPRFIAVGYVDDTQ
FVRFDSDAASQRMEPRAPWIEQEGPEYWDQETRNVKAQSQTDRVDLGTL
RGYYNQSEDGSHTIQIMYGCDVGPDGRFLRGYRQDAYDGKDYIALNEDL
RSWTAADMAAQITKRKWEAAHAAEQQRAYLEGRCVEWLRRYLENGKETL
QRTDPPKTHMTHHPISDHEATLRCWALGFYPAEITLTWQRDGEDQTQDT
ELVETRPAGDGTFQKWAAVVVPSGEEQRYTCHVQHEGLPKPLTLRWELS
SQPTIPIVGIIAGLVLLGAVITGAVVAAVMWRRKSSDRKGGSYTQAASS
DSAQGSDVSLTACKV
GRINA SC HLA-A11
(SEQ ID NO: 31)
MSRSVALAVLALLSLSGLEASIRQAFIRKGGGGSGGGGSGGGGSIQRTP
KIQVYSRHPAENGKSNFLNCYVSGFHPSDIEVDLLKNGERIEKVEHSDL
SFSKDWSFYLLYYTEFTPTEKDEYACRVNHVTLSQPKIVKWDRDMGGGG
SGGGGSGGGGSGGGGSGSHSMRYFYTSVSRPGRGEPRFIAVGYVDDTQF
VRFDSDAASQRMEPRAPWIEQEGPEYWDQETRNVKAQSQTDRVDLGTLR
GYYNQSEDGSHTIQIMYGCDVGPDGRFLRGYRQDAYDGKDYIALNEDLR
SWTAADMAAQITKRKWEAAHAAEQQRAYLEGRCVEWLRRYLENGKETLQ
RTDPPKTHMTHHPISDHEATLRCWALGFYPAEITLTWQRDGEDQTQDTE
LVETRPAGDGTFQKWAAVVVPSGEEQRYTCHVQHEGLPKPLTLRWELSS
QPTIPIVGIIAGLVLLGAVITGAVVAAVMWRRKSSDRKGGSYTQAASSD
SAQGSDVSLTACKV
MZF1 SC HLA-A24
(SEQ ID NO: 32)
MSRSVALAVLALLSLSGLEAKWPPATETLGGGGSGGGGSGGGGSIQRTP
KIQVYSRHPAENGKSNFLNCYVSGFHPSDIEVDLLKNGERIEKVEHSDL
SFSKDWSFYLLYYTEFTPTEKDEYACRVNHVTLSQPKIVKWDRDMGGGG
SGGGGSGGGGSGGGGSGSHSMRYFSTSVSRPGRGEPRFIAVGYVDDTQF
VRFDSDAASQRMEPRAPWIEQEGPEYWDEETGKVKAHSQTDRENLRIAL
RYYNQSEAGSHTLQMMFGCDVGSDGRFLRGYHQYAYDGKDYIALKEDLR
SWTAADMAAQITKRKWEAAHVAEQQRAYLEGTCVDGLRRYLENGKETLQ
RTDPPKTHMTHHPISDHEATLRCWALGFYPAEITLTWQRDGEDQTQDTE
LVETRPAGDGTFQKWAAVVVPSGEEQRYTCHVQHEGLPKPLTLRWEPSS
QPTVPIVGIIAGLVLLGAVITGAVVAAVMWRRNSSDRKGGSYSQAASSD
SAQGSDVSLTACKV
EBV F12 SC HLA-A11
(SEQ ID NO: 33)
MSRSVALAVLALLSLSGLEAAVFDRKSDAKGGGGSGGGGSGGGGSIQRT
PKIQVYSRHPAENGKSNFLNCYVSGFHPSDIEVDLLKNGERIEKVEHSD
LSFSKDWSFYLLYYTEFTPTEKDEYACRVNHVTLSQPKIVKWDRDMGGG
GSGGGGSGGGGSGGGGSGSHSMRYFYTSVSRPGRGEPRFIAVGYVDDTQ
FVRFDSDAASQRMEPRAPWIEQEGPEYWDQETRNVKAQSQTDRVDLGTL
RGYYNQSEDGSHTIQIMYGCDVGPDGRFLRGYRQDAYDGKDYIALNEDL
RSWTAADMAAQITKRKWEAAHAAEQQRAYLEGRCVEWLRRYLENGKETL
QRTDPPKTHMTHHPISDHEATLRCWALGFYPAEITLTWQRDGEDQTQDT
ELVETRPAGDGTFQKWAAVVVPSGEEQRYTCHVQHEGLPKPLTLRWELS
SQPTIPIVGIIAGLVLLGAVITGAVVAAVMWRRKSSDRKGGSYTQAASS
DSAQGSDVSLTACKV
EBV F28 SC HLA-A11
(SEQ ID NO: 34)
MSRSVALAVLALLSLSGLEAIVTDFSVIKGGGGSGGGGSGGGGSIQRTP
KIQVYSRHPAENGKSNFLNCYVSGFHPSDIEVDLLKNGERIEKVEHSDL
SFSKDWSFYLLYYTEFTPTEKDEYACRVNHVTLSQPKIVKWDRDMGGGG
SGGGGSGGGGSGGGGSGSHSMRYFYTSVSRPGRGEPRFIAVGYVDDTQF
VRFDSDAASQRMEPRAPWIEQEGPEYWDQETRNVKAQSQTDRVDLGTLR
GYYNQSEDGSHTIQIMYGCDVGPDGRFLRGYRQDAYDGKDYIALNEDLR
SWTAADMAAQITKRKWEAAHAAEQQRAYLEGRCVEWLRRYLENGKETLQ
RTDPPKTHMTHHPISDHEATLRCWALGFYPAEITLTWQRDGEDQTQDTE
LVETRPAGDGTFQKWAAVVVPSGEEQRYTCHVQHEGLPKPLTLRWELSS
QPTIPIVGIIAGLVLLGAVITGAVVAAVMWRRKSSDRKGGSYTQAASSD
SAQGSDVSLTACKV
HLA-A02
(SEQ ID NO: 35)
GSHSMRYFFTSVSRPGRGEPRFIAVGYVDDTQFVRFDSDAASQRMEPRA
PWIEQEGPEYWDGETRKVKAHSQTHRVDLGTLRGYYNQSEAGSHTVQRM
YGCDVGSDWRFLRGYHQYAYDGKDYIALKEDLRSWTAADMAAQTTKHKW
EAAHVAEQLRAYLEGTCVEWLRRYLENGKETLQRTDAPKTHMTHHAVSD
HEATLRCWALSFYPAEITLTWQRDGEDQTQDTELVETRPAGDGTFQKWA
AVVVPSGQEQRYTCHVQHEGLPKPLTLRWEPSSQPTIPIVGIIAGLVLF
GAVITGAVVAAVMWRRKSSDRKGGSYSQAASSDSAQGSDVSLTACKV
CMV pp65 SC HLA-A02
(SEQ ID NO: 36)
MSRSVALAVLALLSLSGLEANLVPMVATVGGGGSGGGGSGGGGSIQRTP
KIQVYSRHPAENGKSNFLNCYVSGFHPSDIEVDLLKNGERIEKVEHSDL
SFSKDWSFYLLYYTEFTPTEKDEYACRVNHVTLSQPKIVKWDRDMGGGG
SGGGGSGGGGSGGGGSGSHSMRYFFTSVSRPGRGEPRFIAVGYVDDTQF
VRFDSDAASQRMEPRAPWIEQEGPEYWDGETRKVKAHSQTHRVDLGTLR
GYYNQSEAGSHTVQRMYGCDVGSDWRFLRGYHQYAYDGKDYIALKEDLR
SWTAADMAAQTTKHKWEAAHVAEQLRAYLEGTCVEWLRRYLENGKETLQ
RTDAPKTHMTHHAVSDHEATLRCWALSFYPAEITLTWQRDGEDQTQDTE
LVETRPAGDGTFQKWAAVVVPSGQEQRYTCHVQHEGLPKPLTLRWEPSS
QPTIPIVGIIAGLVLFGAVITGAVVAAVMWRRKSSDRKGGSYSQAASSD
SAQGSDVSLTACKV
Cancer SC HLA-A02
(SEQ ID NO: 37)
MSRSVALAVLALLSLSGLEAFLLDGSANVGGGGSGGGGSGGGGSIQRTP
KIQVYSRHPAENGKSNFLNCYVSGFHPSDIEVDLLKNGERIEKVEHSDL
SFSKDWSFYLLYYTEFTPTEKDEYACRVNHVTLSQPKIVKWDRDMGGGG
SGGGGSGGGGSGGGGSGSHSMRYFFTSVSRPGRGEPRFIAVGYVDDTQF
VRFDSDAASQRMEPRAPWIEQEGPEYWDGETRKVKAHSQTHRVDLGTLR
GYYNQSEAGSHTVQRMYGCDVGSDWRFLRGYHQYAYDGKDYIALKEDLR
SWTAADMAAQTTKHKWEAAHVAEQLRAYLEGTCVEWLRRYLENGKETLQ
RTDAPKTHMTHHAVSDHEATLRCWALSFYPAEITLTWQRDGEDQTQDTE
LVETRPAGDGTFQKWAAVVVPSGQEQRYTCHVQHEGLPKPLTLRWEPSS
QPTIPIVGIIAGLVLFGAVITGAVVAAVMWRRKSSDRKGGSYSQAASSD
SAQGSDVSLTACKV
HLA-A24
(SEQ ID NO: 40)
MAVMAPRTLVLLLSGALALTQTWAGSHSMRYFSTSVSRPGRGEPRFIAV
GYVDDTQFVRFDSDAASQRMEPRAPWIEQEGPEYWDEETGKVKAHSQTD
RENLRIALRYYNQSEAGSHTLQMMFGCDVGSDGRFLRGYHQYAYDGKDY
IALKEDLRSWTAADMAAQITKRKWEAAHVAEQQRAYLEGTCVDGLRRYL
ENGKETLQRTDPPKTHMTHHPISDHEATLRCWALGFYPAEITLTWQRDG
EDQTQDTELVETRPAGDGTFQKWAAVVVPSGEEQRYTCHVQHEGLPKPL
TLRWEPSSQPTVPIVGIIAGLVLLGAVITGAVVAAVMWRRNSSDRKGGS
YSQAASSDSAQGSDVSLTACKV

### Example 1

Generation of Artificial Antigen-Presenting Cells

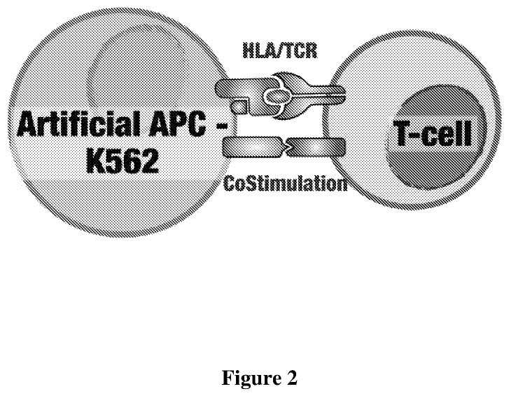

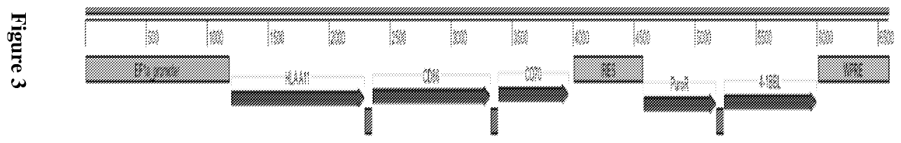

Development of T cell-based immunotherapy requires the generation or expansion of antigen-specific T cells. K562 aAPC cells can be used for the stimulation or expansion of antigen-specific T cells. This is done by first loading the K562 aAPC cells with a peptide antigen and co-culturing these aAPCs with T cells. Antigen-specific T cells are stimulated to expand through the peptide/HLA complex as well as the engagement of the co-stimulatory molecules (FIG. 2).

Optimal stimulation of T cells requires the engagement of T cell receptors (TCR) and also interactions between co-stimulatory molecules and their ligands. Engagement of the T cell receptor provides specificity against particular antigens whereas the co-stimulatory molecules enhance proliferation and/or augment the phenotype of the T cell. There are multiple classes of co-stimulatory molecules which contribute to the activation of T cells. Although the role of individual co-stimulatory molecules like CD28 (ligands CD80 and CD86) are well established, how combinations of these co-stimulatory molecules work together to influence the activation and phenotype of T cells is unclear.

To circumvent the need to generate moDCs from individuals and have an off-the-shelf approach to enrich or expand antigen-specific T cells, artificial antigen-presenting cells (aAPCs) expressing HLA-A11 and co-stimulatory ligands were generated. In addition, aAPCs expressing HLA-A02 and co-stimulatory ligands were also generated. K562 cell line was transduced with a lentiviral particle encoding HLA-A11 and the co-stimulatory ligands CD86, CD70 and CD137L (UniProtKB ID: P42081, P32970 and P41273, respectively). Stable expression of the HLA and co-stimulatory ligands was selected by antibiotic selection and sorting for cells that have high expression of these surface markers.

### Example 2

Expansion of Shared Antigen-directed T cells for treatment of patients or functional analysis Frequency of antigen-specific T cells is typically very low in patients or after in-vitro stimulation using monocyte derived dendritic cells (moDC). Due to the low frequency and presence of T cells with different antigen specificity, enrichment and further expansion of these antigen-specific T cells is required for treatment of patients or functional analysis. Expansion of antigen-specific T cells requires the use of antigen-presenting cells that selectively promote the growth of T cells that recognize an antigen that is being presented.

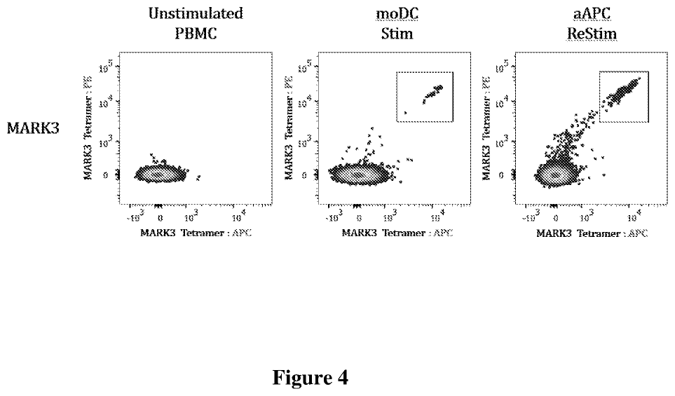

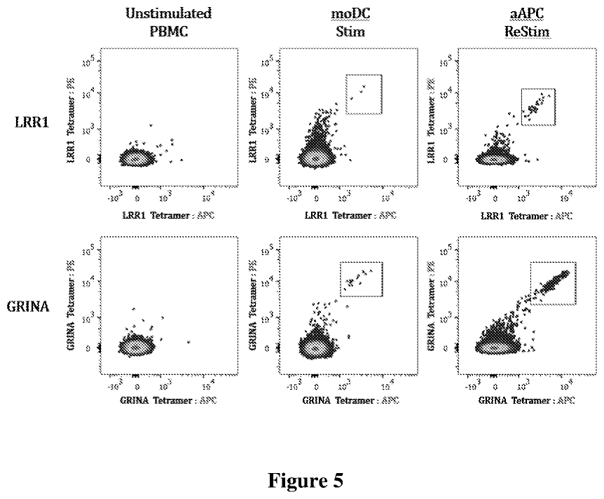

Currently, antigen-specific T cells are routinely generated by co-culturing monocyte-derived dendritic cells (moDC) with T cells. FIGS. 4 and 5 demonstrate the use of moDC for the initial generation of antigen-specific T cells against antigens (in this case, splice variant antigens like MARK3, LRR1 and GRINA). Monocyte-derived dendritic cells from healthy donors (HSA 27 and 38) were generated from isolated CD14 positive monocytes (Human CD14 Positive Selection Kit, STEMCELL Technologies). Briefly, differentiation of monocytes to dendritic cells was carried out by culturing the isolated CD14 cells with IL4 (10 ng/ml) and GM-CSF (800 IU/ml) for 3 days and maturating the dendritic cells with IL4 (10 ng/ml), GM-CSF (800 IU/ml), LPS (10 ng/ml), IFN-γ (100 IU/ml), and the MARK3, LRR1, or GRINA HLA-A11 SVP (5 μM) overnight.

These monocyte-derived dendritic cells were then cultured with CD8+ T cells which were isolated from another aliquot of PBMCs from the same donor using EasySep™ CD8+ T cell isolation kit, STEMCELL Technologies. The co-culture period can be anywhere between, for example, 7 days and 12 days, depending on the strength of the stimulation and the amount of growth observed. In this example, after 11 days of co-culture, expansion of antigen-specific T cells was detected by staining with tetramers (labeled with PE and APC) that had been loaded with the MARK3, LRR1, or GRINA HLA-A11 SVP (middle column in FIGS. 4 and 5).

Further expansion of antigen-specific T cells was performed by co-culturing these T cell cultures with the K562 artificial antigen-presenting cells. K562 artificial antigen-presenting cells were first treated with mitomycin to prevent outgrowth of these cells, and then loaded with SVPs (5 μM MARK3, LRR1, or GRINA peptide) before co-culturing with T cells. Cytokine cocktails used during the co-culture: Interleukin 21 (30 ng/ml) was added during the initial 3 days; and Interleukin 7 and Interleukin 15 (5 ng/ml each) were used subsequently. After co-culture, antigen-specific T cells against SVPs were detected by staining the cells with SVP tetramers. Further expansion of antigen against SVP was observed as shown in FIGS. 4 and 5 (right vs middle column). Similarly, further expansion of antigen-specific T cells may be performed by substituting the aAPCs with the SC HLA aAPC, as described in Example 6 below.

Treatment of patients may be carried out by expansion of splice variant antigen-specific T cells from the patient in the manner just described, and administering these expanded T cells back into the patient. The quantity of T cells to be used for treatment can be increased by using a larger amount of starting material and/or by expansion with aAPCs as shown below in Example 3. In addition, the quantity of antigen-specific T cells may alternatively be expanded using SC HLA aAPC, as is described below in Example 6 or Example 10. In either case, T cells that have been obtained from a patient may be engineered to express TCR sequences that confer specificity to the desired antigen, as discussed again below in Example 7.

Alternatively, these antigen-specific T cells can be used for further functional testing such as identification of antigen-specific TCRs or detecting an immune response to antigens.

### Example 3

Generation and Further Expansion of Antigen-Specific T Cells Using Artificial APCs

Unlike the activation of antigen-specific memory T cells, which are present at greater frequency and primed for mounting an immune response, the primary response to antigens requires the activation of naïve antigen-specific T cells which requires additional signals. In vivo, dendritic cells (DC) provide factors that can activate naïve T cells whereas other antigen-presenting cells like B cells are unable to do so. The procedures for generating DCs in vitro are complicated and tedious (as described in Example 2). Furthermore, it is challenging to generate sufficient antigen-specific T cells from naïve T cells.

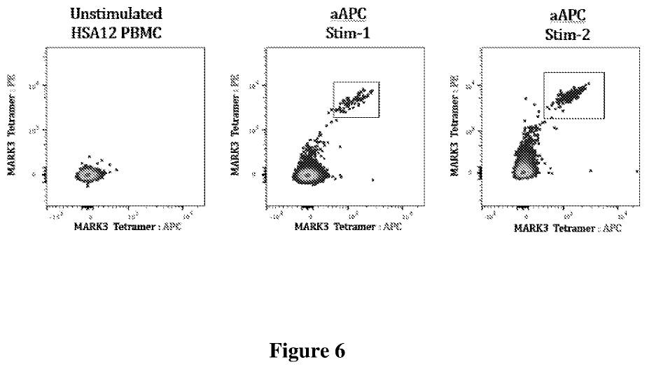

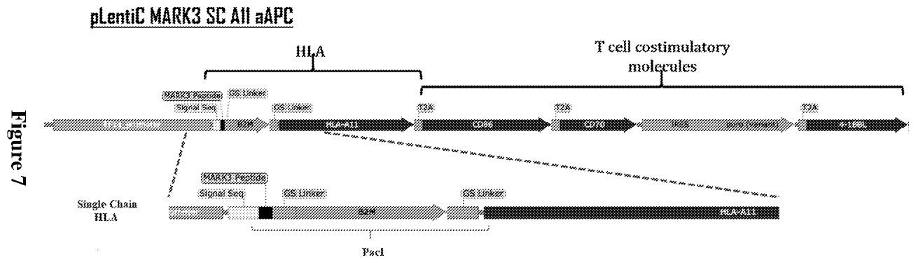

Antigen-specific T cells against MARK3 SVP, generated from naïve CD8+ T cells using the K562 artificial antigen-presenting cell, is shown in FIG. 6. Briefly, K562 artificial antigen-presenting cells were first treated with mitomycin to prevent outgrowth of these cells, and then loaded with 5 μM MARK3 SVPs before co-culturing with naïve CD8+ T cells. Naïve CD8+ T cells were isolated by depletion of memory T cell markers (EasySep™ Human Naïve CD8+ T Cell Isolation Kit II, STEMCELL Technologies) from a healthy donor (HSA 12). Cytokine cocktail was used during the co-culture: Interleukin 21 (30 ng/ml) was added during the initial 3 days; and Interleukin 7 and Interleukin 15 (5 ng/ml each) were used subsequently. After co-culture, antigen-specific T cells against SVPs were detected by staining the cells with SVP tetramers. Repeating co-culture of T cells with the aAPC loaded with MARK3 SVP leads to the further expansion of MARK3 SVP specific T cells as shown in FIG. 6 (enrichment of MARK3 specific T cells from 0.11% to 0.56% CD8+ T cells).

### Example 4

aAPCs can be Used to Screen for Potential Antigens

A cell-based approach for the identification of antigens using these aAPCs may be developed. K562 cells retain the ability to process antigens internally (cleave and load peptide(s) onto HLA molecules). This property can be utilized to screen for antigens by expressing sequences that are potentially antigenic in these K562 cells. Alternatively, peptides that are suspected of being antigenic might be loaded onto these aAPCs. These aAPCs presenting the antigen to be tested are then subsequently co-cultured with T cells. T cells that recognize the antigen can be determined by an IFN-γ Elispot assay or tetramer staining. The IFN-γ Elispot assay is based on the detection of IFN-γ secretion by T cells recognizing their target via TCR/HLA interactions.

### Example 5

Generation of Single Chain (SC) HLA Artificial Antigen-Presenting Cells

Antigen presentation by HLA is an intrinsic property of most cells, including professional antigen-presenting cells like macrophages or dendritic cells. Antigen presentation begins in the ER where HLA binds peptides (typically 8-11 amino acid long) generated from protein turnover. These HLA/peptide complexes are then presented on the surface of antigen-presenting cells allowing T cells to survey and identify cells that have been transformed or infected. However, due to the large number of peptides generated from proteins present in the cell, peptides compete for binding to HLA and only peptides with high affinity binding to HLA will be presented on HLA. This creates a situation where the amount of any one peptide presented by HLA on the surface of the antigen-presenting cell may be limited.

Artificial APCs expressing a HLA binding peptide in cis with the HLA (SC HLA) greatly increase the density of the antigen that is presented on the surface and hence provide stronger stimulation to particular antigen-specific T cells. This is particularly true for priming naïve T cells where a critical density of antigens on the cell surface and additional co-stimulation is required for activation. Furthermore, multiple SC HLA aAPCs can be used to generate antigen-specific T cells against multiple targets (see Example 6, below). This is highly advantageous and provides an off-the-shelf approach to generating antigen-specific T cells, especially in scenarios where the number of T cells is limited.

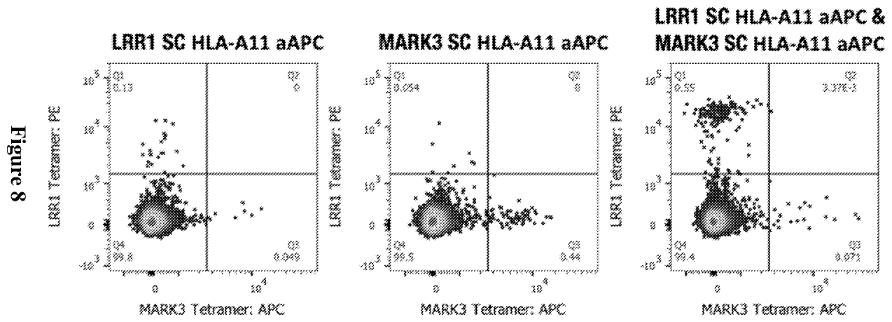

The single chain HLA-A11 construct (for description of SC-HLA constructs see https://www.jimmunol.org/content/168/7/3145) comprises sequences that encode: 1) HLA binding peptide; 2) linkers; 3) beta2-microglobulin (B2M); and 4) HLA, as shown in FIG. 7.

Artificial APCs expressing SC HLA and co-stimulatory ligands were generated using a lentiviral vector, shown in FIG. 7. K562 cell line was transduced with a lentiviral particle encoding SC HLA-A11 (SEQ ID NO: 18) and the co-stimulatory ligands CD86, CD70 and CD137L (UniProtKB ID: P42081, P32970 and P41273, respectively). Stable expression of the HLA and co-stimulatory ligands was selected by antibiotic selection and sorting for cells that had high expression of these surface markers.

Single chain HLA-A11 for different antigens can be made by changing the sequence of the HLA binding peptide. SC HLA has been generated for MARK3, LRR1, EBV F12, EBV F28, and CAMKK1.

Additionally, the HLA used in a SC construct can be any HLA. SC HLA has also been generated, for example, for HLA-A02 and HLA-A24.

### Example 6

Generation of Antigen-Specific T Cells Using SC HLA aAPCs

T cells with their cognate TCR sequences recognizing specific targets are typically rare, and it is challenging to identify or isolate a particular population of antigen-specific T cells. Selective detection and/or expansion of these antigen-specific T cells would greatly facilitate their identification and/or use for therapy. Furthermore, the immunogenicity may not be clear for different antigens, or there may be a limited amount of material available for generating antigen-specific T cells. Hence, it would be desirable to be able to stimulate CD8+ T cells with antigen-presenting cells that present particular different antigens. Currently, multiple antigen-specific T cells in a sample can be generated by adding multiple peptides to antigen-presenting cells and co-culturing them with T cells. However, these exogenous peptides might compete for binding to the same HLA, leading to limited presentation of these peptide antigens and, hence, may not provide optimal stimulation of desired antigen-specific T cells.

In accordance with the present invention, antigen-specific T cells against LRR1 and MARK3 were generated using LRR1 and MARK3 SC HLA aAPCs. Briefly, K562 single chain HLA artificial antigen-presenting cells were first treated with mitomycin to prevent outgrowth of these cells, and co-cultured with naïve CD8+ T cells. Naïve CD8+ T cells were isolated by depletion of memory T cell markers (EasySep™ Human Naïve CD8+ T Cell Isolation Kit II, STEMCELL Technologies) from a healthy donor. Cytokine cocktail used during the co-culture: Interleukin 21 (30 ng/ml) was added during the initial 3 days; and Interleukin 7 and Interleukin 15 (5 ng/ml each) were used subsequently. Co-culture of LRR1 SC HLA-A11 aAPC, MARK3 SC HLA-A11 aAPC, and both LRR1 SC HLA-A11 aAPC and MARK3 SC HLA-A11 aAPC, with naïve CD8+ T cells was done to determine whether antigen-specific T cells could be generated using the SC HLA aAPCs. After co-culture, the cells were stained with tetramers loaded with LRR1 or MARK3 peptide (tetramers were either labelled with PE or APC, respectively). Antigen-specific T cells against LRR1 or MARK3 were indeed detected, as shown in FIG. 8. Co-culture of SC HLA-A11 aAPC with naïve CD8+ T cells leads to the expansion of its respective specific antigen, as shown in FIG. 8. For example, co-culture of naïve CD8+ T cells with LRR1 SC HLA-A11 aAPC leads to expansion of LRR1-specific CD8+ T cells (Frequency of LRR1-specific T-cells is 0.13%; left panel), compared to co-culture with MARK3 SC HLA-A11 aAPC (Frequency of LRR1-specific T-cells is 0.054%; middle panel). Furthermore, when naïve CD8+ T cells were co-cultured with LRR1 SC HLA-A11 aAPC and MARK3 SC HLA-A11 aAPC, both LRR1 and MARK3 antigen-specific T cells could be detected (Frequency of LRR- and MARK3-specific T cells is 0.55% and 0.071%, respectively; right panel). This shows that the SC HLA aAPC can specifically expand desired antigen-specific T cells and multiple antigen-specific T cells can be generated by co-culturing with multiple SC HLA aAPCs. These antigen-specific T cells can subsequently be used for TCR identification.

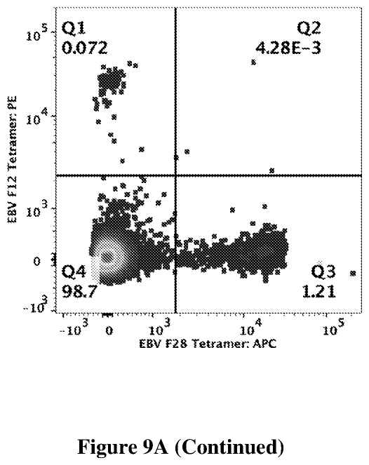

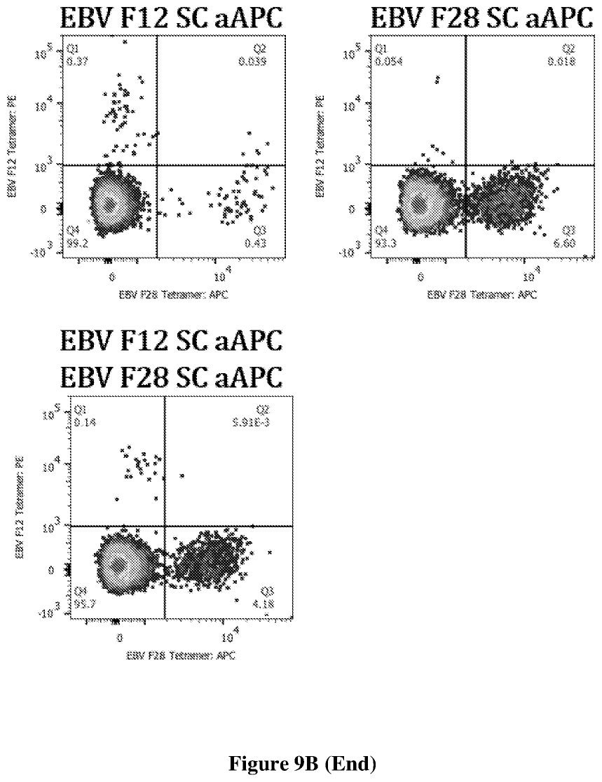

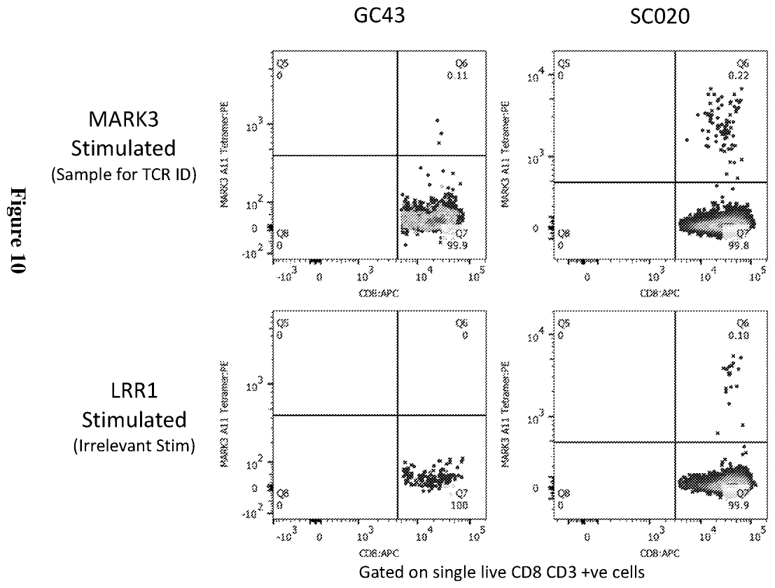

In another example, antigen-specific T cells against EBV F12 and EBV F28 were expanded using EBV F12 and EBV F28 SC HLA aAPC. Tetramer staining for antigen-specific T cells against EBV F12 and F28 using an aliquot of PBMC from a healthy donor (HSA29) was performed to determine whether this donor had an immune response to EBV and what was the frequency/functionality of these antigen-specific T cells in the donor (as further described in Example 8). PBMC from this donor were stained with tetramers loaded with EBV F12 or EBV F28 peptide and labelled with PE and APC, respectively. The frequency of antigen-specific T cells in this donor is shown in FIG. 9A.

Co-culture of EBV F12 SC HLA-A11 aAPC, EBV F28 SC HLA-A11 aAPC, and both EBV F12 SC HLA-A11 aAPC and EBV F28 SC HLA-A11 aAPC with total CD8+ T cells was done to determine whether antigen-specific T cells could be expanded using the SC HLA aAPC and whether these antigen-specific T cells were functional (as described in Example 8). Briefly, K562 single chain HLA artificial antigen-presenting cells were first treated with mitomycin to prevent outgrowth of these cells, and co-cultured with total CD8+ T cells. Total CD8+ T cells were isolated (EasySep™ CD8+ T cell isolation kit, STEMCELL Technologies) from a PBMC donor. Cytokine cocktail was used during the co-culture: Interleukin 21 (30 ng/ml) was added during the initial 3 days; and Interleukin 7 and Interleukin 15 (5 ng/ml each) were used subsequently. After co-culture, the cells were stained with tetramers loaded with EBV F12 or EBV F28 peptide and antigen-specific T cells against EBV F12 or EBV F28 could indeed be detected, as shown in FIG. 9B. As can be seen, co-culture of SC HLA-A11 aAPC with total CD8+ T cells leads to the expansion of its respective specific antigen. For example, co-culture of total CD8+ T cells with EBV F12 SC HLA-A11 aAPC leads to expansion of EBV F12 specific CD8+ T cells (Frequency of EBV F12 specific T-cells is 0.37%; top left column) as does co-culture with EBV F28 SC HLA-A11 aAPC (Frequency of EBV F12 specific T-cells is 0.054%; right panel). Furthermore, when total CD8+ T cells were co-cultured with both EBV F12 SC HLA-A11 aAPC and EBV F28 SC HLA-A11 aAPC, both EBV F12 and EBV F28 antigen-specific T cells could be detected (Frequency of EBV F12 and EBV F28 T-cells is 0.14% and 4.18%, respectively; bottom left column). This is an increase in the frequency of these antigen-specific T cells when compared to the frequency present in the donor prior to co-culture (frequency of EBV F12 and EBV F28 specific T-cells is 0.072% and 1.21%, respectively, as shown in FIG. 9A). This shows that the SC HLA aAPC can specifically expand desired antigen-specific T cells and multiple antigen-specific T cells can be generated by co-culturing with multiple SC HLA aAPCs. These antigen-specific T cells can subsequently be used for TCR identification. These EBV-specific T cells can also be used for treatment of chronic EBV infection.

### Example 7

Generation of Antigen-Specific T Cells for Treatment

Antigen-specific T cells can be used for the treatment of patients with viral disease and/or cancer. The treatment of patients with T cells may comprise the following steps: 1) confirmation of antigen expression in patient; 2) screening for T cell responses to target antigens in patient; 3) obtaining T cells from patient and/or donor (for example, PBMCs or tumour infiltrating lymphocytes from patient); 4) ex-vivo expansion of antigen-specific T cells using the relevant aAPC of the present invention; and 5) infusion of expanded T cells into patients for treatment of disease. Additionally, T cells that have been obtained from a patient and/or donor may be engineered to express TCR sequences that confer specificity to the desired antigen.

Patients requiring treatment with T cells may require screening for T cell response to antigen as described below in Example 8, prior to subsequent expansion of antigen-specific cells. For example, in patients who have undergone organ transplant, reactivation of latent EBV infection may occur due to immunosuppression. The PBMCs from these patients would need to be tested for T cell responses to EBV antigens to determine whether: 1) they have immune responses to EBV; and/or 2) antigen-specific T cells might be generated from them in sufficient quantities for therapy.

Patients having recurrent/refractory or metastatic cancer may express a splice variant antigen (such as MARK3, LRR1 and/or GRINA), and they may have low number of antigen-specific T cells that recognize these antigens. A sample of cancerous tissue may be tested for the expression of the splice variant antigen to determine whether patients will benefit from the treatment with antigen-specific T cells. This may be done by RT-PCR. The patient may also be tested for the expression of the relevant HLA type, such as HLA-A11 for MARK3.

The relevant antigen-specific T cells may then be expanded as described in Examples 3 or 6. Additionally antigen-specific T cells for multiple different antigens may be manufactured simultaneously using the SC HLA aAPC as described in Example 6. PBMCs used for manufacturing T cells for therapy can be derived either from the patient or from HLA-matched healthy donors. For example, EBV-specific T cells can be generated/expanded from a healthy donor that has similar HLA haplotype to a patient requiring treatment with EBV-specific T cells. Matching the HLA haplotype of the healthy donor and the patient minimizes the risk of graft vs host disease.

Prior to administering these T cells, the patient may be treated with cyclophosphamide and fludarabine.

### Example 8

Screening for T Cell Responses to Antigen

During oncogenesis or viral infection, antigens are expressed in the target cell and these antigens contain multiple HLA binding peptides. What peptide(s) is presented by HLA is dependent on the HLA haplotype of the individual. Determining whether a particular peptide is immunogenic or not is challenging due to peptides competing for binding to HLA. Similarly, T cell responses might be skewed towards particular peptides due to frequency or properties of the responding T cell. For example, T cells may lose their ability to respond to antigens after long term exposure to antigen. It is essential not only to identify whether a particular peptide is being presented by the cell, but also to determine T cell responses to particular peptides. Knowledge of the T cell responses to antigen may help guide the treatment of patients or development of products. The use of aAPC to screen for immune response to cancer antigen removes the need to prepare antigen presenting cells from the patient. Additionally using the SC HLA aAPC further removes the need to separately load antigen and facilitates testing immune responses to multiple antigens as was explained in Example 6.

EBNA 3B is an antigen, present in the EBV genome, which produces immune responses depending on the HLA type. There are two peptides that can bind to HLA-A11 and different individuals may respond differently to these antigens. To determine which of these peptides a person is responding to and what is the T cell response of the donor, frequency of EBV-specific T cells was detected directly ex-vivo and after co-culture with EBV F12 and F28 SC HLA-A11 aAPC (as described in Example 6). An immune response to both peptides was detected prior to co-culture with SC HLA aAPC (as shown in FIG. 9A) and frequency of antigen-specific T cells increased in response to antigen stimulation by its respective SC HLA aAPC (FIG. 9B). This knowledge may then be used as a guide for the treatment of disease or manufacture of immunotherapy products. For example, proliferation of antigen-specific T cells in response to antigen stimulation shows that the T cells are not exhausted, and antigen-specific T cells might be manufactured from the donor/patient.

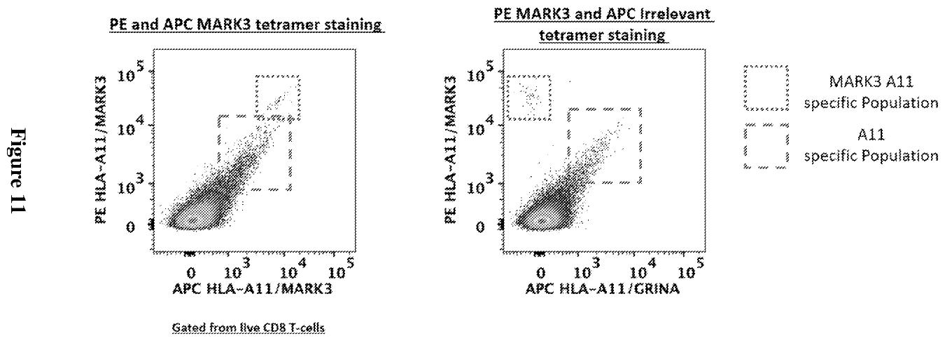

Similarly, immune response to antigens present in other diseases like cancer can be determined this way as shown in FIG. 10. For example, cancer patients expressing SVP antigens like MARK3 or LRR1 antigen might not have a robust immune response to these antigens, and therefore sufficient numbers of antigen-specific T cells may not be able to be generated from their PBMCs or tumour infiltrating lymphocytes.

MARK3 was previously found to be aberrantly spliced in gastric cancer and some patients have T cell responses to a splice variant peptide derived from this aberrant splicing event. To determine whether gastric cancer patients (GC43 and SC020) have T cell responses to MARK3 SVP, PBMCs (GC43) or CD8+ T cells (SC020) were co-cultured with the MARK3 SC aAPC and MARK3-specific T cells identified using tetramers labelled with PE and loaded with MARK3 peptide. Using this method, a small quantity of patient PBMC or CD8+ T cells can be used and allows a functional readout of the immune response to the antigen.

For GC patient (SC020), the experiment was performed by isolating total CD8+ T cells (EasySep™ CD8 T cell isolation kit, STEMCELL Technologies) from PBMCs that were collected post-surgery. This patient was previously shown to have MARK3-specific T cells before surgery and it was desired to determine whether the patient continued to have an immune response to this antigen after surgery. MARK3 SC aAPC or LRR1 SC aAPC (irrelevant antigen stimulation) were co-cultured with these isolated CD8+ T cells and Interleukin 21 (30 ng/ml) was added during the initial 3 days; subsequently media containing Interleukin 2 (5 ng/ml), Interleukin 15 (5 ng/ml) and Interleukin 21 (30 ng/ml) was added every other day. On day 9, the presence of MARK3-specific T cells was determined by tetramer staining (FIG. 10).

Similarly, immune response to MARK3 was determined in gastric cancer patient GC43 using co-culture of PBMCs with MARK3 SC aAPC or LRR1 SC aAPC (irrelevant antigen stimulation). In this case, CD8+ T cells were not isolated as only a very limited amount of PBMCs was available (1.24 million PBMCs). Immune response to MARK3 could be detected for both patients, i.e.: expansion of MARK3-specific T cells could be observed in the sample that was co-cultured with the MARK3 SC aAPC compared to LRR1 SC aAPC (top vs bottom row for samples from each patient; FIG. 10). Furthermore, increase in number of MARK3-specific T cells observed in gastric cancer patient SC020 shows that these cells are functional, i.e.: they proliferate in response to stimulation with the MARK3 SVP antigen, and suggests that sufficient quantities of MARK3-specific T cells might be generated for treatment in this patient.

### Example 9

Generation of Antigen-Specific T Cells by Non-Self HLA Using aAPC

During the maturation of the immune system, survival of a T cell is based on its threshold of TCR recognition of self HLA, i.e.: TCR sequences of the T cell must be able to bind to self HLA to a certain degree, but not have too high an affinity against self-antigen/HLA. T cells that do not meet these requirements will not survive. This determines the TCR repertoire present in different individuals. Therefore, generation of antigen-specific T cells in a HLA mismatched donor situation would yield a different or greater diversity of TCR sequences with different properties that may be advantageous. Co-culture of T cells and APCs that are HLA-mismatched results in the generation of (i) T cells that recognize foreign HLA independently of the peptide that is bound by the HLA, as well as (ii) T cells that specifically recognize antigen/HLA complex. The cells from (i) are not desired for the purposes of the present invention. Using an artificial antigen-presenting cell that presents a single peptide antigen and HLA greatly facilitates the generation of antigen-specific T cells in a HLA mismatched setting.

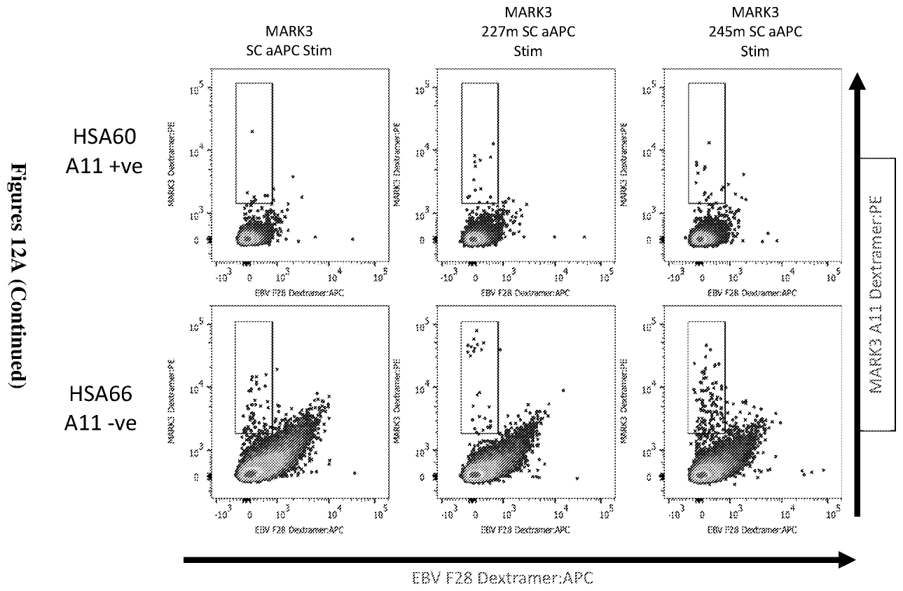

MARK3 specific T-cells were generated in a HLA mismatched donor using MARK3 SC HLA-A11 aAPC. Briefly, total CD8+ T cells were isolated from a HLA mismatched donor and co-cultured with the MARK3 SC HLA-A11 aAPC (as described in the EBV F12/F28 in Example 6). Tetramer staining was used to determine the specificity of the antigen-specific CD8+ T cells that recognize: 1) HLA-A11 irrespective of peptide bound; or 2) MARK3/HLA-A11. CD8+ T cells from the co-culture were used for tetramer staining (Staining 1: MARK3-loaded HLA-A11 tetramer that was labelled with either PE or APC, Staining 2: MARK3-loaded HLA-A11 tetramer that was labelled with PE, and GRINA-loaded HLA-A11 tetramer that was labelled with APC). FACS data for this is shown in FIG. 11. CD8+ T cells that specifically recognize MARK3 peptide are shown by dotted boxes; these cells are stained only by tetramers loaded with MARK3 peptide and not tetramers loaded with GRINA (FACS data on right), whereas CD8+ T cells that recognize HLA-A11 independently of peptide are shown by the dashed boxes. These CD8+ T cells are stained by HLA-A11 tetramers irrespective of the peptide that is bound by the HLA-A11 tetramer (both FACs data). This demonstrates that the SC HLA-A11 aAPC can be used to generate antigen-specific T-cells in a HLA mismatched donor.

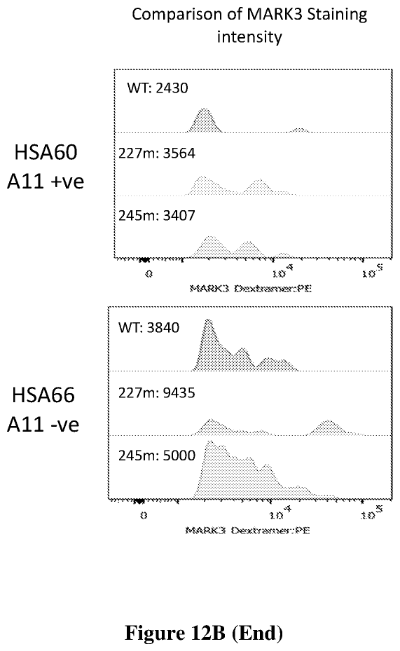

Furthermore, as shown in FIG. 12B, MARK3-specific T cells generated using the SC HLA aAPC in a HLA mismatched donor (HSA66) show greater staining intensity for the MARK3 dextramer when compared to a HLA matched donor (HLA60). This may suggest differences in affinity/avidity for its target, i.e.: T cells generated in the HLA mismatched donor (HSA66) may have greater affinity for the target.

### Example 10

Generation/Selection of Antigen-Specific T Cells Using SC HLA aAPCs with Altered CD8 Binding.

T cell activation is regulated by engagement of the multi-subunit TCR signaling complex. TCR binding to the peptide/HLA complex is one of the key steps in activation of TCR signaling and the strength of this interaction determines whether a T cell is activated or not. The affinity and specificity of the TCR is the key determinant of whether individual T cell clones respond to antigens.

Co-receptor molecules like CD8 and CD4 are also part of the TCR signaling complex and can augment TCR signaling through recruitment of the LCK kinase, a component of the TCR signaling complex. Additionally, CD8, through binding to a conserved region in HLA class I, increases the avidity of the TCR peptide/HLA interaction and stabilizes the interaction between the TCR and peptide/HLA complex. Using HLA class I molecules with decreased CD8 binding increases the threshold of TCR affinity required for activation of T cells. Using these mutant HLA with decreased CD8 binding to present antigen on antigen presenting cells therefore allows selection of T cells with higher affinity for the peptide/HLA complex.

MARK3 HLA-A11 SC aAPC with mutant HLA that have decreased CD8 binding and co-stimulatory ligands were generated in a manner similar to that described in Example 5. The mutant HLA contain mutations, 227 m and 245 m, in the alpha-3 immunoglobulin domain which abrogate or diminish binding to the CD8 co-receptor (Dutoit V et. al.).

Briefly, naïve CD8+ T cells were isolated from PBMCs from two healthy donors HSA60 and HSA66 who were HLA-A11 positive and negative, respectively. Experiments were undertaken to generate antigen-specific T cells for MARK3 for both donors by co-culturing naïve CD8+ T cells separately with MARK3 SC HLA-A11 aAPCs or with the aAPC variants that have mutations for CD8 binding (227 m and 245 m). The procedure was as described in Example 6.

Antigen-specific T cells for MARK3 were generated in HLA matched and mismatched settings for the PBMCs from healthy donors HSA60 and HSA66, respectively. After co-culture for 7 days, antigen-specific T cells were identified using dextramer staining. PE and APC labelled dextramer loaded with MARK3 and EBV F28 peptide, respectively, were used to determine the antigen specificity of the CD8+ T cells. T cells recognizing the MARK3 SVP will only be labelled with the PE labelled tetramer; this is particularly important for antigen-specific T cells generated in the HLA mismatched donor due to the risk of cross-reactivity to HLA as explained in Example 9 (FIG. 12). As can be seen in FIG. 12A, antigen-specific T cells for MARK3 were generated when naïve CD8+ T cells from HSA60 and HSA66 were co-cultured with MARK3 SC aAPC and its variants (indicated by boxes in FIG. 12A). MARK3-specific T cells generated in the same donor (either HSA60 or HSA66) show differences in MARK3 dextramer staining intensity, as shown in FIG. 12B. This demonstrates that although the aAPC presents the same MARK3 SVP, there is selective expansion of particular clonotypes of T cells when co-cultured with naïve CD8+ T cells from the same donor. With the SC HLA aAPC variants (227 m and 245 m), T cells with greater MARK3 dextramer staining intensity were expanded when compared to co-cultures with MARK3 SC aAPC. For example, geometric means of MARK3 dextramer staining for MARK3-specific T cells generated in HSA66 using the MARK3 SC HLA 227 m aAPC and MARK3 SC HLA aAPC (WT) were 9435 and 3840, respectively (FIG. 12B). This demonstrates the selective expansion of antigen-specific T cells and provides a method for isolating T cells with different affinity/avidity for its target for subsequent applications, such as TCR isolation.

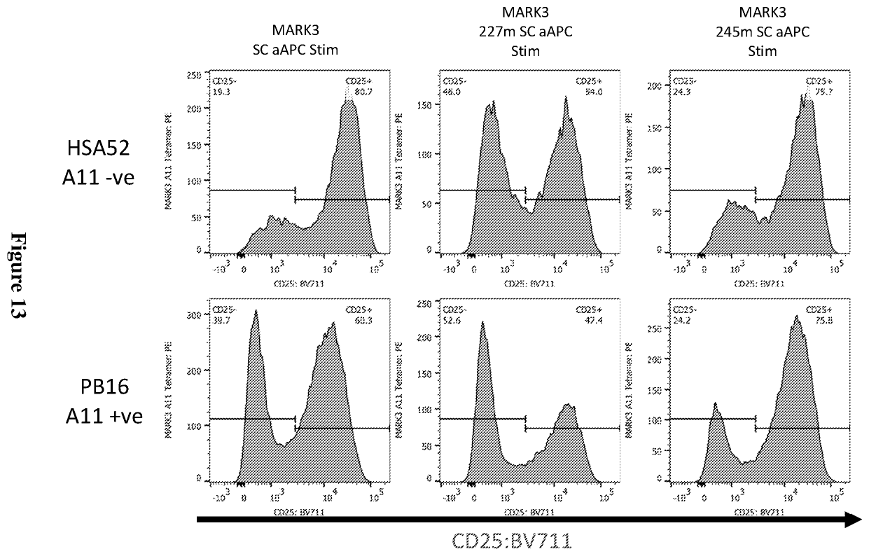

Additionally, CD8+ T cells co-cultured with the 227 m mutant SC aAPC show a less activated T cell phenotype when compared to the original MARK3 SC aAPC, which is observed for both donors HSA60 and HSA66. This is likely to do with the strength of TCR activation using the 227 m HLA, as it has been shown (Dutoit V et. al.) that this mutant HLA cannot bind to CD8 and would therefore provide a weaker stimulus for T cell activation compared to wild type HLA. This is shown in FIG. 13. CD25 is a marker that is expressed in activated T cells and staining of cells with an antibody recognizing CD25 shows that after 7 days of co-culture, a large population of CD8+ T cells expressed this marker. CD8+ T cells that have been co-cultured with MARK3 227 m SC aAPC show decreased numbers of CD8+ T cells that express CD25 compared to co-cultures with MARK3 SC aAPC (middle and left column of FIG. 13).

### Example 11

Increased Expansion of Antigen-Specific T Cells Using aAPC Compared to moDC

The frequency of antigen-specific T cells in PBMCs is typically very low, and expansion of these rare antigen-specific T cells is required for any downstream applications. It was observed that expansion of antigen-specific T cells using the aAPC expressing CD86, CD70 and CD137L (as described in Example 2) is as efficient at inducing antigen-specific T cells as using moDC. (0.12% and 0.11% MARK3 specific T cells were generated using moDC and aAPCs as shown in FIGS. 4 and 6, respectively). Artificial APCs expressing the single chain HLA and the co-stimulatory ligands (as described in Example 5) further increase the amount of antigen-specific T cells that can be obtained. As shown in FIG. 8, 0.44% of MARK3 specific T cells were generated with the MARK3 SC HLA aAPC (middle panel of FIG. 8). This is much more than the 0.12% of MARK3 specific T cells obtained using moDC (middle panel of FIG. 4).

As mentioned in Example 3, naïve CD8+ T cells require additional signals for activation. In the experiments using aAPCs, naïve CD8+ T cells were used to generate antigen-specific CD8+ T cells. It was shown that the combination of co-stimulatory ligands was able to activate and subsequently expand antigen-specific T cells from the naïve pool of CD8+ T cells. Inclusion of the SC HLA increases the efficiency at which antigen-specific T cells can be generated.

### REFERENCES

Butler MO, et al. 2007, Clin Cancer Res 13(6):1857-67;
Cranage et al. 1986, EMBO J. 5:3057-3063;
Dutoit V, et al, 2003, J Immunol. 170(10):5110-7;
Remington's Pharmaceutical Sciences, 18th Ed. Mack Printing Company, 1990, pp. 1289-1329;
WO94/17810, published Aug. 18, 1994; and
WO94/23744, published Oct. 27, 1994.

1. An artificial antigen-presenting cell (aAPC) comprising at least one immune stimulatory ligand and co-stimulatory ligands comprising or consisting of CD86, CD70 and CD137L, wherein the aAPC is capable of inducing proliferation and/or expanding an immune cell population when contacted with the aAPC.

2. The aAPC of claim 1, wherein the aAPC comprises a recombinant nucleic acid encoding at least one immune stimulatory ligand and co-stimulatory ligands comprising or consisting of CD86, CD70 and CD137L, optionally wherein the recombinant nucleic acid further encodes an antigen.

3. (canceled)

4. The aAPC of claim 2, wherein the recombinant nucleic acid encodes a fusion protein comprising an antigen and an immune stimulatory ligand.

5. The aAPC of claim 1, wherein the immune stimulatory ligand is a major histocompatibility complex (MHC) polypeptide, optionally wherein the MHC polypeptide is a MHC class I or a MHC class II polypeptide.

6. (canceled)

7. The aAPC of claim 5, wherein the MHC polypeptide is loaded with an antigen.

8. The aAPC of claim 1, wherein the aAPC is a mammalian cell, optionally wherein the aAPC is a K562 cell or HEK293T cell.

9. (canceled)

10. The aAPC of claim 1, wherein the immune cell is a T cell.

11. The aAPC of claim 1, wherein the immune stimulatory ligand is modified to have attenuated binding affinity to CD8.

12. (canceled)

13. An isolated nucleic acid encoding a) a fusion protein comprising an immune stimulatory ligand and an antigen and b) co-stimulatory ligands comprising or consisting of CD86, CD70 and CD137L.

14. A vector comprising the isolated nucleic acid of claim 13, optionally wherein the vector is a viral vector.

15. (canceled)

16. A method of preparing the aAPC of claim 1, wherein the method comprises transfecting or transducing a mammalian cell with the isolated nucleic acid of claim 13.

17. A method for inducing proliferation of an immune cell, said method comprising contacting said immune cell with the aAPC of claim 1, thereby inducing proliferation of the immune cell.

18. A method for expanding a population of antigen-specific immune cells, said method comprising contacting a population of immune cells with the aAPC of claim 1, thereby expanding the population of antigen-specific immune cells.

19. The method of claim 18, wherein the method comprises contacting the population of immune cells with two or more distinct aAPCs to expand two or more distinct populations of antigen-specific immune cells.

20. A method of generating a population of antigen-specific immune cells, said method comprising contacting a population of immune cells with the aAPC of claim 1 to generate the population of antigen-specific immune cells.

21. The method of claim 20, wherein the method comprises contacting the population of immune cells with two or more distinct aAPCs to generate two or more distinct populations of antigen-specific immune cells.

22. (canceled)

23. A method for inducing an immune cell response to an antigen in a subject, the method comprising administering the aAPC of claim 1 to the subject, wherein the aAPC induces proliferation of an immune cell specific for the antigen, thereby inducing an immune cell response to the antigen in the subject.

24. A method of treating a medical condition in a subject, the method comprising:
a) expanding a population of immune cells that has been isolated from the subject by contacting the population of immune cells with the aAPC of claim 1; and
b) administering the expanded population of immune cells to the subject to treat the medical condition in the subject.

25. A method of identifying an antigenic peptide in a subject, the method comprising:
a) contacting the aAPC of claim 1 with a population of immune cells that has been obtained from the subject, wherein the aAPC is loaded with a candidate peptide or comprises a recombinant nucleic acid encoding a candidate peptide; and
b) detecting a population of immune cells that recognizes the candidate peptide, thus identifying the candidate peptide as an antigenic peptide,
optionally wherein the population of immune cells that recognizes the candidate peptide is detected with an ELISPOT assay and/or enumeration of antigen-specific T cells.

26. A method for identifying or detecting the presence of an immune cell that recognizes an antigen, said method comprising contacting said immune cell with one or more aAPCs of claim 1 that presents said antigen, and identifying or detecting the presence of a population of immune cells that recognizes said antigen, optionally wherein the population of immune cells that recognizes the antigen is detected with an ELISPOT assay and/or enumeration of antigen-specific T cells.

27. (canceled)
